(19) United States
(12) Patent Application Publication
(10) Pub. No.: US 2020/0208102 A1
(43) Pub. Date: Jul. 2, 2020

(54) DRIED TISSUE MODEL

(71) Applicant: TOHOKU UNIVERSITY, Sendai-shi, Miyagi (JP)

(72) Inventors: Makoto OHTA, Sendai-shi, Miyagi (JP); Yasutomo SHIMIZU, Sendai-shi, Miyagi (JP); Taihei ONISHI, Sendai-shi, Miyagi (JP)

(73) Assignee: TOHOKU UNIVERSITY, Sendai-shi, Miyagi (JP)

(21) Appl. No.: 16/617,849

(22) PCT Filed: Jun. 4, 2018
(86) PCT No.: PCT/JP2018/021381

§ 371 Date: Nov. 27, 2019

(30) Foreign Application Priority Data
Jun. 2, 2017 (JP) 2017-110128

(51) Int. Cl. C12N 5/00

(52) U.S. Cl. CPC C12N 5/0062; C12N 2533/30; C12N 2513/00; C12N 5/0068

(57) ABSTRACT

A dried tissue model includes a dried hydrogel which is a dried product of a hydrogel composition, in which when a solvent with which the dried hydrogel can be impregnated is used as a second solvent and the dried hydrogel is impregnated with the second solvent to form an impregnated hydrogel, the impregnated hydrogel satisfies at least one of the following: the impregnated hydrogel has a modulus of elasticity in shear of 0.9×10² to 2.1×10² kPa; the impregnated hydrogel has a viscosity of 4.8 to 17.6 kPa; and the ratio of a second Young's modulus at a strain of 0.5 of the impregnated hydrogel to a first Young's modulus at a strain of 0.5 of the hydrogel composition is 0.39 to 0.69.

### TECHNICAL FIELD

The present invention relates to a dried tissue model produced using a hydrogel composition which is used as a material for molding a tissue model and to a solvent impregnation method for the dried tissue model.

### BACKGROUND ART

As an example of a hydrogel composition which is used as a molding material of a tissue model, PTL 1 discloses a poly(vinyl alcohol) hydrogel composition.

### CITATION LIST

### Patent Literature

PTL 1: JP-A-2007-316434

### SUMMARY OF INVENTION

### Technical Problem

The poly(vinyl alcohol) hydrogel illustrated in PTL 1 has a property that a moisture content decreases over time in normal temperature. It is known that, when the moisture content of the polyvinyl alcohol hydrogel decreases, the polyvinyl alcohol hydrogel is dried so that physical and mechanical properties such as elasticity or viscosity are lost. Thus, in a tissue model using the polyvinyl alcohol hydrogel of PTL 1, as the moisture content of the polyvinyl alcohol hydrogel increases, a period of time for which the tissue model can be used after being manufactured is shortened.

Further, a method for preventing a decrease in the moisture content of a polyvinyl alcohol hydrogel to extend the time period for which a tissue model can be used includes, for example, storing the tissue model in a sealed state at a cold dark place such as a refrigerator. However, in the case of manufacturing an organ model or a biological model as a tissue model, the size of the tissue model becomes large, which makes it difficult to store the tissue model in a sealed state at a cold dark place.

As described above, in the tissue model produced using the polyvinyl alcohol hydrogel of PTL 1, there is a problem in that an improvement in convenience of use is difficult.

Furthermore, in the case of the tissue model of PTL 1 being dried, since there is no specific means for restoring physical properties of the polyvinyl alcohol hydrogel, reuse of the tissue model is difficult as a matter of practice. Thus, in the tissue model produced using the polyvinyl alcohol hydrogel of PTL 1, there is a problem in that, in the case of the tissue model being dried, the tissue model has to be discarded so that time and cost for manufacturing a new tissue model are required.

The present invention has been made in order to solve the above-described problems, and an object thereof is to provide a dried tissue model by which convenience of use of a tissue model can be improved and time and cost for manufacturing a new tissue model can be reduced.

In addition, another object of the present invention is to provide a solvent impregnation method for the above-described dried tissue model by which convenience of use of a tissue model can be improved and time and cost for manufacturing a new tissue model can be reduced.

### Solution to Problem

A dried tissue model of the present invention is a dried tissue model, including a dried hydrogel which is a dried product of a hydrogel composition, the hydrogel composition including a polyvinyl alcohol resin having a three-dimensional network structure, and a first solvent which is confined in a network portion of the three-dimensional network structure to lose fluidity, in which when a solvent with which the dried hydrogel can be impregnated is used as a second solvent and the dried hydrogel is impregnated with the second solvent to form an impregnated hydrogel, the impregnated hydrogel has a modulus of elasticity in shear of 0.9×10² to 2.1×10² kPa.

In addition, the present invention is directed to a dried tissue model including a dried hydrogel which is a dried product of a hydrogel composition, the hydrogel composition including a polyvinyl alcohol resin having a three-dimensional network structure and a first solvent which is confined in a network portion of the three-dimensional network structure to lose fluidity, in which when a solvent with which the dried hydrogel can be impregnated is used as a second solvent and the dried hydrogel is impregnated with the second solvent to form an impregnated hydrogel, the impregnated hydrogel has a viscosity of 4.8 to 17.6 kPa.

Furthermore, the present invention is directed to a dried tissue model including a dried hydrogel which is a dried product of a hydrogel composition, the hydrogel composition including a polyvinyl alcohol resin having a three-dimensional network structure and a first solvent which is confined in a network portion of the three-dimensional network structure to lose fluidity, in which when a solvent with which the dried hydrogel can be impregnated is used as a second solvent and the dried hydrogel is impregnated with the second solvent to form an impregnated hydrogel, the ratio of a second Young's modulus at a strain of 0.5 of the impregnated hydrogel to a first Young's modulus at a strain of 0.5 of the hydrogel composition is 0.39 to 0.69.

Furthermore, the present invention is directed to a solvent impregnation method for a dried tissue model, the method including a step of immersing the dried tissue model in a second solvent at a predetermined temperature so that the dried tissue model can be impregnated with the second solvent.

### Advantageous Effects of Invention

According to the present invention, the tissue model can be dried and stored. In addition, by immersing the dried tissue model in the solvent, it is possible to remanufacture a tissue model including an impregnated hydrogel having desired physical properties.

Therefore, according to the present invention, it is possible to provide a dried tissue model by which convenience of use of a tissue model can be improved and time and cost for manufacturing a new tissue model can be reduced.

Furthermore, according to the present invention, it is possible to provide a solvent impregnation method for the above-described dried tissue model by which convenience of use of a tissue model can be improved and time and cost for manufacturing a new tissue model can be reduced.

### BRIEF DESCRIPTION OF DRAWINGS

FIG. 1 is a chemical formula representing a structure of polyvinyl alcohol constituting a hydrogel composition of the present invention.

FIG. 2 is a schematic view illustrating a chemical structure of the hydrogel composition of the present invention.

FIG. 3 is a schematic view illustrating a part of a crosslink region by a hydrogen bond of the hydrogel composition of the present invention.

FIG. 4 is a graph showing a change over time in weight in a sample of a dried hydrogel according to Example 3 of the present invention.

FIG. 5 is a graph showing a change over time in weight ratio in the sample of the dried hydrogel according to Example 3 of the present invention with respect to a sample of a hydrogel composition.

FIG. 6 is a graph showing a change over time in solvent evaporated weight in the sample of the dried hydrogel according to Example 3 of the present invention.

FIG. 7 is a graph showing a change over time in solvent evaporated weight per hour in the sample of the dried hydrogel according to Example 3 of the present invention.

FIG. 8 is a graph showing a change over time in weight ratio in a sample of an impregnated hydrogel according to Example 4 of the present invention with respect to a sample of a hydrogel composition.

FIG. 9 is a graph showing a change over time in weight per hour in the sample of the impregnated hydrogel according to Example 4 of the present invention.

FIG. 10 is a graph showing a stress-strain curve of the hydrogel composition and the impregnated hydrogel of the present invention.

FIG. 11 is a graph showing a relation between a temperature and a dynamic viscoelasticity in an impregnated hydrogel according to Example 6 of the present invention.

FIG. 12 is a graph showing a correlation of a dynamic viscoelasticity in the impregnated hydrogel according to Example 6 of the present invention.

FIG. 13 is a graph showing an average value of Young's moduli in repeated remanufacturing of an impregnated hydrogel according to Example 7 of the present invention.

### DESCRIPTION OF EMBODIMENTS

### [Chemical Structure of Polyvinyl Alcohol 1]

A chemical structure of polyvinyl alcohol 1 constituting a hydrogel composition of the present invention will be described.

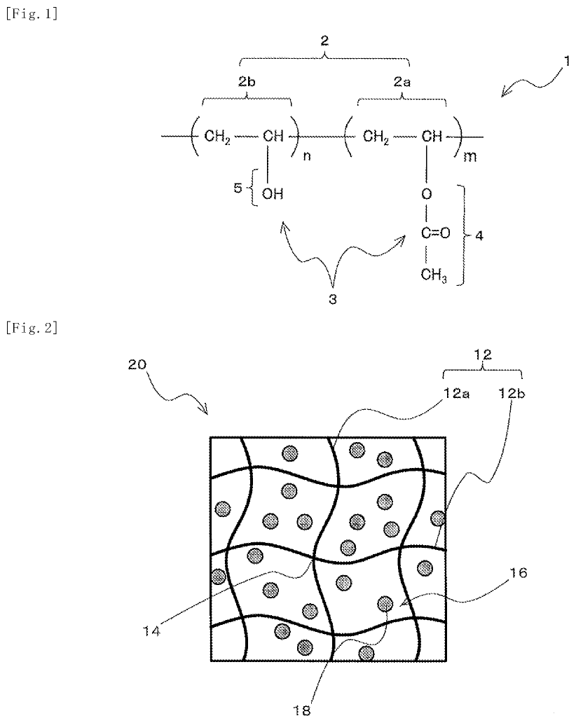

FIG. 1 is a chemical formula representing the structure of the polyvinyl alcohol 1 constituting the hydrogel composition of the present invention. Incidentally, m and n in the chemical formula of FIG. 1 represent variables indicating an integer of 1 or more.

As illustrated in FIG. 1, the polyvinyl alcohol 1 is a synthetic resin of a polymer compound composed of a linear basic skeleton 2 and functional groups 3. The basic skeleton 2 of the polyvinyl alcohol 1 is composed of m first hydrocarbon skeleton parts 2a represented by chemical formula —CH₂—CH— and n second hydrocarbon skeleton parts 2b represented by the same chemical formula —CH₂—CH— as that of the first hydrocarbon skeleton part 2a. In the polyvinyl alcohol 1, the first hydrocarbon skeleton part 2a and the second hydrocarbon skeleton part 2b are linearly and randomly bonded to each other by a covalent bond between carbon atoms. The functional group 3 has m hydrophobic acetate groups 4 (—COOCH₃), one of which is linked to one first hydrocarbon skeleton part 2a, and n hydrophilic hydroxyl groups 5 (—OH), one of which is linked to one second hydrocarbon skeleton part 2b.

### [Method for Producing Polyvinyl Alcohol 1]

Next, an example of a method for producing the polyvinyl alcohol 1 as an industrial product will be described.

The polyvinyl alcohol 1 is produced by a process including the steps of synthesizing and purifying a vinyl acetate monomer from ethylene and acetic acid, polymerizing the vinyl acetate monomer to produce a polymer, polyvinyl acetate, and saponifying the polyvinyl acetate to replace some of the acetate groups 4 of the polyvinyl acetate by hydroxyl groups 5.

### (Synthesis Step)

First, the step of synthesizing and purifying a vinyl acetate monomer will be described.

The vinyl acetate monomer is synthesized by an oxidative dehydrogenation reaction using ethylene, acetic acid, and oxygen as starting materials as represented in the following Chemical Reaction Formula (1). The synthesis method is also referred to as ethylene method.

CH₂=CH₂+CH₃COOH+½O₂→CH₂=CHOCHCO₃+H₂O (1)

In the synthesis of vinyl acetate, a gas phase method of subjecting ethylene gas, acetic acid gas, and oxygen gas to an oxidative dehydrogenation reaction in the presence of a supported catalyst is used. The oxidative dehydrogenation reaction is generally performed in a fixed-bed catalytic reactor, but may be performed in a fluidized-bed catalytic reactor. As the fixed-bed catalytic reactor, a multitubular reactor in which a supported catalyst is filled in a catalyst-filled tube provided inside the reactor is used. A reactor is designed by a material having a large thermal conductivity in order to prevent the activity of the supported catalyst from being reduced by heat generated by the oxidative dehydrogenation reaction. For example, the reactor can be made of stainless steel.

Further, as the supported catalyst, for example, a catalyst in which metallic palladium as a main catalyst is supported on a carrier and potassium acetate as an auxiliary catalyst serving as a reaction accelerator is supported on the carrier is used. As the carrier supporting the main catalyst and the auxiliary catalyst, for example, a porous material such as alumina, silica, activated carbon, or titania is used. The amount of the main catalyst supported on the carrier can be set, for example, to be 0.1 to 1.0 wt % in the case of metallic palladium. In addition, the amount of the auxiliary catalyst supported on the carrier can be set, for example, to be 0.5 to 5.0 wt % in the case of potassium acetate. Further, the supported catalyst may be a catalyst in which a metal such as gold, copper, or cadmium as a secondary auxiliary catalyst is further supported on the carrier.

In the reactor, a mixture gas containing a vinyl acetate monomer is produced. The mixture gas containing a vinyl acetate monomer is cooled in a heat exchanger and separated into a liquid phase component containing a vinyl acetate monomer and a gas phase component containing a by-product such as carbon dioxide in a separator. The liquid phase component containing a vinyl acetate monomer is subjected to fractional distillation in a distillation column to purify the vinyl acetate monomer.

### (Polymerization Step)

Next, the polymerization step to produce a polymer, polyvinyl acetate, will be described.

The polymer, polyvinyl acetate, is produced by dissolving the vinyl acetate monomer purified in the synthesis process in a methanol solvent and performing solution polymerization in the methanol solution of the vinyl acetate. The solution polymerization is performed by a radical polymerization reaction to produce the polymer, polyvinyl acetate, having a desired degree of polymerization.

The radical polymerization reaction is one of polymerization reactions by chemical reaction species in which neutral radical species having high activity are used as growth species and the radical species are subjected to an addition reaction to double bond of a vinyl compound so as to subject the vinyl compound to an addition reaction. The radical polymerization reaction is performed by adding a small amount of a radical polymerization initiator, which causes a radical polymerization reaction by decomposition in response to heat or light, to a methanol solution and is controlled to produce polyvinyl acetate having a desired degree of polymerization.

Incidentally, the degree of polymerization is one of parameters that determine physical properties of the polyvinyl alcohol 1, and details thereof will be described later.

A temperature condition of the radical polymerization reaction varies also depending on a desired degree of polymerization of polyvinyl acetate, the type of the radical polymerization initiator, or the like, and thus is not limited; however, the radical polymerization reaction can be performed in a temperature range of −30° C. to 150° C. For example, the radical polymerization reaction can be performed in a temperature range of 0° C. to 100° C. In addition, the radical polymerization reaction is typically performed in normal pressure, but may be performed under pressure. Furthermore, regarding the radical polymerization reaction, the radical polymerization reaction by heat is first advanced, and then the radical polymerization reaction by light is advanced, so that the radical polymerization reaction can be controlled to produce polyvinyl acetate having a desired degree of polymerization.

As the radical polymerization initiator, an azo compound such as azobisisobutyronitrile or azobisisobutyric acid ester, an inorganic peroxide such as potassium persulfate, or an organic peroxide such as benzoyl peroxide is used. A radical polymerization initiator, which is considered to be suitable for solution polymerization of vinyl acetate, is benzoyl peroxide or azobisisobutyronitrile. Incidentally, the radical polymerization initiator may be prepared such that a reaction rate of the radical polymerization reaction is controlled by using two or more kinds of radical polymerization initiators in combination and polyvinyl acetate having a desired degree of polymerization can be produced.

Further, an organic acid may be added to the methanol solution in order to control the radical polymerization reaction of producing polyvinyl acetate to be performed at a desired reaction rate, and thus the vinyl acetate may be prevented from being hydrolyzed. As the organic acid, for example, tartaric acid, citric acid, acetic acid, and the like are used.

Further, a chain transfer agent may be added to the methanol solution to adjust the degree of polymerization of polyvinyl acetate to be a desired degree of polymerization. As the chain transfer agent, for example, 2-mercaptoethanol, acetaldehyde, and the like are used.

Incidentally, the vinyl acetate, the methanol solution, the radical polymerization initiator, the organic acid, and the chain transfer agent which are described above are subjected to a deoxygenation treatment before the radical polymerization reaction in order to avoid oxygen inhibition in the radical polymerization reaction.

A polymerization apparatus used in the above polymerization process may include, for example, a mixing tank in which a mixed liquid is produced by mixing vinyl acetate, methanol solution, and a radical polymerization initiator and a reaction tank in which the vinyl acetate in the mixed liquid is radically polymerized with light or heat. Incidentally, the polymerization apparatus is not limited to the apparatus having the above configuration, and it will be understood that a polymerization apparatus with any configuration other than the above may also be used as long as it can be generally used for subjecting a vinyl compound monomer to a radical polymerization reaction by solution polymerization to produce a vinyl compound polymer.

### (Saponification Step)

Next, the saponification step in which some of the acetate groups 4 of the polyvinyl acetate are replaced by hydroxyl groups 5 will be described.

The saponification is a chemical reaction in which an acid or alkali is added to a compound having an ester bond to hydrolyze the compound into a salt and an alcohol. In the saponification process, by adding alkali such as sodium hydroxide to the polymer, polyvinyl acetate, in the methanol solvent, some of the acetate groups 4 of the polyvinyl acetate are replaced by hydroxyl groups 5 so that polyvinyl alcohol 1 having a desired degree of saponification is produced. The polyvinyl alcohol 1 produced by saponification is precipitated in the methanol solvent and aggregated along with impurities such as sodium acetate.

Incidentally, the degree of saponification is one of parameters that determine physical properties of the polyvinyl alcohol 1 along with the aforementioned degree of polymerization, but the details thereof will be described later along with the degree of polymerization.

A temperature condition in which the saponification is performed varies also depending on a desired degree of saponification of the polyvinyl alcohol 1 and a time for performing the saponification, and thus is not limited; however, the saponification can be performed in a temperature range of 30 to 60° C. For example, the saponification can be performed in a temperature range of 35 to 60° C., 40 to 60° C., or 40 to 42° C. Further, the time for performing the saponification varies also depending on a desired degree of saponification of the polyvinyl alcohol 1 and the temperature condition in which the saponification is performed, and thus is not limited; however, the saponification can be performed, for example, in a range of 2 to 20 hours.

The alkali to be added in the saponification is not limited to sodium hydroxide. For example, the alkali to be added in the saponification may be, for example, hydroxides of alkali metals such as potassium hydroxide and lithium hydroxide or hydroxide compounds of quaternary ammonium such as tetraethylammonium hydroxide. Further, the saponification can also be performed, for example, by adding an acid such as hydrochloric acid or sulfuric acid instead of an alkali. Further, after completion of the saponification, the added alkali or acid may be subjected to a neutralization reaction.

The aggregate of the polyvinyl alcohol 1 is pulverized as necessary and then separated from the methanol solvent by a solid-liquid separation treatment such as centrifugal separation. The precipitate of the polyvinyl alcohol 1 separated from the methanol solvent is washed with purified water such as ion-exchange water or distilled water and thus impurities such as sodium acetate contained in the precipitate of the polyvinyl alcohol 1 are separated in water. By performing the solid-liquid separation treatment such as centrifugal separation again, the precipitate of the polyvinyl alcohol 1 is separated from water and then purified to thereby produce crystalline polyvinyl alcohol 1. The crystalline polyvinyl alcohol 1 is dried to remove moisture, thereby obtaining a dried product of the polyvinyl alcohol 1.

The precipitate of the polyvinyl alcohol 1 may be washed with an alcohol such as ethanol instead of purified water. Further, a temperature condition for washing varies also depending on a desired upper limit of sodium acetate residue and thus is not limited; however, the washing can be performed in a temperature range of 30 to 60° C. For example, the washing can be performed in a temperature range of 35 to 55° C. or 40 to 42° C. Further, a washing time varies also depending on a desired upper limit of sodium acetate residue and thus is not limited; however, the washing time can be set to 1 to 10 hours. Further, the number of times of the washing may be only once or may be one or more times as necessary. Further, stirring at the time of the washing may or may not be performed.

A saponification apparatus used in the above-described saponification process can be configured, for example, to include a mixer in which a mixed liquid obtained by mixing a methanol solution of polyvinyl acetate and sodium hydroxide is produced and a saponification reactor in which the polyvinyl acetate in the mixed liquid is saponified. Incidentally, the saponification apparatus is not limited to the apparatus having the above-described configuration. For example, it is obvious that a saponification apparatus can be configured, as necessary, to include a solid-liquid separator such as a centrifugal separator or a dehydrator, a pulverizer, a stirrer, a washer, a dryer, or the like which is generally used for saponifying a polymer compound having an ester bond.

### (Other Processes)

Incidentally, in production of the polyvinyl alcohol 1 as an industrial product, a recovery process of purifying and recovering the methanol solvent or sodium acetate separated by the solid-liquid separation treatment or the washing treatment and then reusing the methanol solvent or sodium acetate is performed separately from the synthesis process, the polymerization process, and the saponification process described above. For example, methanol can be purified by distillation from a mixed liquid of sodium acetate and methanol and can be recovered as methanol gas. The recovered methanol gas is cooled and then reused as a methanol solvent in the polymerization process. Further, the sodium acetate is reacted with a strong acid such as hydrochloric acid or sulfuric acid, and the produced acetic acid is distilled and thus can be recovered as acetic acid gas. The acetic acid gas is reused as a starting material in the synthesis process of vinyl acetate.

### (Modified Example of Method for Producing Polyvinyl Alcohol 1)

Hereinbefore, an example of the method for producing the polyvinyl alcohol 1 as an industrial product has been described, but the polyvinyl alcohol 1 may be produced by a synthesis process, a polymerization process, and a saponification process which are different from those described above.

For example, in the synthesis process, the vinyl acetate may be synthesized by a gas phase method in which acetylene gas and acetic acid gas are used as starting materials and reacted under a catalyst supporting zinc acetate on activated carbon, or the like.

Further, the polyvinyl alcohol 1 can also be produced by using commercially available vinyl acetate as a starting material without the synthesis process of vinyl acetate being performed. Incidentally, in the case of using commercially available vinyl acetate, since impurities such as a polymerization inhibitor that is phenothiazine or hydroquinone are contained in the vinyl acetate, before the radical polymerization reaction, a distilling and purifying treatment of removing impurities by distillation and purifying vinyl acetate is performed.

Further, a polymerization method in the polymerization process is not limited only to the solution polymerization, but polymerization methods such as bulk polymerization, solution polymerization, suspension polymerization, and emulsion polymerization can be used. Further, a solvent in the solution polymerization is not limited only to methanol, but alcohols such as ethanol, butanol, i-propanol, and n-propanol; ketones such as acetone and methyl ethyl ketone; toluene; benzene; and the like can be used. Further, in the polymerization process, vinyl acetate, a radical polymerization initiator, an organic acid, and a chain transfer agent may be added all at once or continuously, or may be added sequentially according to a progress degree of polymerization.

Further, in the saponification process, sodium hydroxide may be added all at once or continuously, or may be added sequentially according to a progress degree of saponification.

### [Industrial Use of Polyvinyl Alcohol 1]

The dried product of the polyvinyl alcohol 1 purified by the above-described production method is subjected to pulverization processing as necessary, and is used as a granular or powdery industrial product.

The polyvinyl alcohol 1 is used as a molding material for manufacturing a tissue model, and is also used as a raw material for a biocompatible material such as artificial joint, a cosmetic raw material, and a pharmaceutical additive.

In addition to the above-described use application, the polyvinyl alcohol 1 can be used in various use applications. For example, the polyvinyl alcohol 1 is also used as a raw material of vinylon that is a synthetic fiber, a raw material of a polarizer film that is an optical film for a flat panel display, and a raw material of an acetal resin. Other than, the polyvinyl alcohol 1 is also used as a fiber processing agent, a coating agent for paper processing, a binder for paper processing, an adhesive, a liquid glue, slime used in toys or science educational materials, a polymerization stabilizer of vinyl chloride, a binder of an inorganic substance, and the like.

### [Chemical Structure of Hydrogel Composition 10]

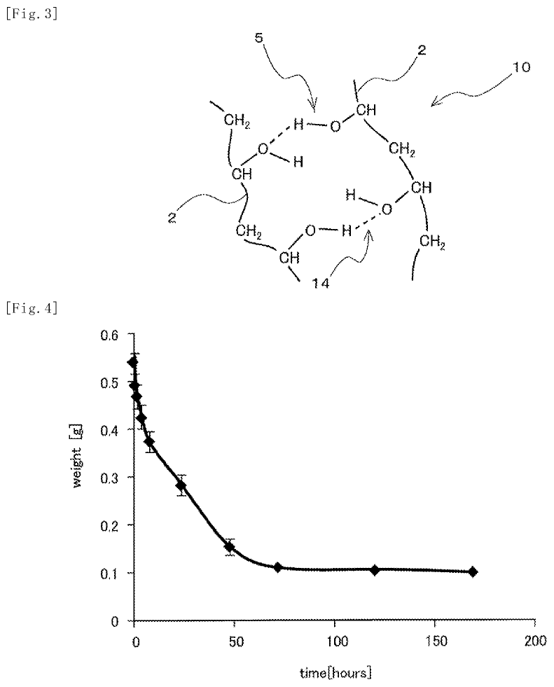

Next, a chemical structure of the hydrogel composition 10 of the present invention will be described using FIG. 2.

FIG. 2 is a schematic view illustrating a chemical structure of the hydrogel composition 10 of the present invention. Incidentally, the hydrogel composition 10 of the present invention contains a resin of polyvinyl alcohol 1 and is also referred to as a polyvinyl alcohol hydrogel. Further, the polyvinyl alcohol hydrogel is also abbreviated as PVA-H.

The gel composition refers to a composition in which, in specific atoms or atom groups contained in chain-like polymer compounds, the polymer compounds are partially bonded to form a three-dimensional network structure and which has a low-molecular-weight solvent, which is confined to lose fluidity, inside the three-dimensional network structure. In the present invention, the gel composition having water inside the network structure as a solvent or the gel composition having a mixed solvent of water and an organic solvent which is miscible in water inside the network structure as a solvent is referred to as the “hydrogel composition.”

As illustrated in FIG. 2, the network structure of the hydrogel composition 10 has polymer chains 12, which are carbon chain moieties having carbon atoms linearly bonded in the polymer compound, and a crosslink region 14 that is a bonding region between first and second polymer chains 12a and 12b. Incidentally, although not illustrated in FIG. 2, the second polymer chain 12b is adjacent to the first polymer chain 12a at a skew position and is sterically bonded in the crosslink region 14. In other words, the three-dimensional network structure of the hydrogel composition 10 is configured by the crosslink region 14 bonding the first polymer chain 12a and the second polymer chain 12b.

Further, as illustrated in FIG. 2, the hydrogel composition 10 contains a low-molecular-weight solvent 18 which is confined in a network portion 16 of the three-dimensional network structure configured by the polymer chain 12 and the crosslink region 14 to lose fluidity. Although not illustrated in the drawing, since the molecule of the solvent 18 receives a strong attracting force, caused by an intermolecular force, from the polymer chain 12, the degree of freedom of the molecule of the solvent 18 becomes lowest in the vicinity of the three-dimensional network structure. That is, a constraint force of the molecule of the solvent 18 becomes largest in the vicinity of the three-dimensional network structure. Further, as the molecule of the solvent 18 is separated away from the three-dimensional network structure, the degree of freedom of the molecule of the solvent 18 is increased and the constraint force of the molecule of the solvent 18 is decreased.

In the hydrogel composition 10 of the present invention, the low-molecular-weight solvent 18 is not limited, but for example, a mixed solvent of water and an organic solvent which is miscible in water, water, or saline can be used.

As water which is preferable used for the solvent 18 of the present invention, for example, pure water such as ion-exchange water, ultrafiltration water, reverse osmosis water, or distilled water, ultrapure water, and the like are mentioned, but the water is not limited thereto.

Further, as the organic solvent which is miscible in water, although not limited, for example, it is possible to use alkyl alcohols having 1 to 4 carbon atoms such as methyl alcohol, ethyl alcohol, n-propyl alcohol, isopropyl alcohol, n-butyl alcohol, sec-butyl alcohol, and tert-butyl alcohol; amides such as dimethylformamide and dimethylacetamide; ketones or ketone alcohols such as acetone, methyl ethyl ketone, and diacetone alcohol; ethers such as tetrahydrofuran and dioxane; polyhydric alcohols such as ethylene glycol, propylene glycol, 1,2-propanediol, 1,2-butanediol, 1,3-butanediol, 1,4-butanediol, diethylene glycol, triethylene glycol, 1,2,6-hexanetriol, thioglycol, hexylene glycol, and glycerin; polyalkylene glycols such as polyethylene glycol and polypropylene glycol; lower alcohol ethers of polyhydric alcohol such as ethylene glycol monomethyl ether, ethylene glycol monoethyl ether, diethylene glycol methyl ether, diethylene glycol ethyl ether, triethylene glycol monomethyl ether, and triethylene glycol monoethyl ether; alkanolamines such as monoethanolamine, diethanolamine, and triethanolamine; N-methyl-2-pyrolidone; 2-pyrolidone; 1,3-dimethyl-2-imidazolidinone; dimethyl sulfoxide; and the like. The above-described organic solvent can be selected depending on purposes such as providing of moisture-retaining property, providing of antibacterial property, providing of antifungal property, providing of conductive property, and adjusting of viscosity or elasticity. Further, as the above-described organic solvent, any one kind of organic solvents may be used singly or two or more kinds of organic solvents may be used.

As an organic solvent which is preferably used in the present invention, for example, there is mentioned acetone, dimethylformamide, glycerin, dimethyl sulfoxide, or the like. An organic solvent which is most preferably used in the present invention is dimethyl sulfoxide which is capable of providing proper elasticity to the hydrogel composition 10. Further, the dimethylformamide is also abbreviated as DMF.

### [Bonding Aspect of Crosslink Region 14 of Hydrogel Composition 10]

Next, a bonding aspect of the crosslink region 14 of the hydrogel composition 10 of the present invention will be described. Hereinafter, in order to describe the bonding aspect of the crosslink region 14, for convenience sake, the description will be given while types of gel composition are roughly classified into a chemical gel and a physical gel.

### (Chemical Gel)

The chemical gel is obtained by crosslinking polymer compounds by a covalent bond and is also referred to as a strongly bonded gel or a chemically crosslinked gel. The chemical gel has characteristics that, since the strength of the crosslinking bond is strong and there is no case where the crosslinking bond is broken due to the molecular motion of the polymer compound by thermal energy, the three-dimensional network structure of the gel composition is not changed by thermal energy.

### (Chemical Crosslinking)

Crosslinking in the chemical gel, that is, chemical crosslinking can be performed, for example, although not limited, by a method of mixing a crosslinking agent such as glutaraldehyde to perform reaction and thereby bonding polymer compounds. Further, other than the above-described method, the chemical crosslinking can also performed, for example, a method of bonding polymer compounds by irradiation of light including radiation such as gamma rays. Further, the chemical crosslinking can also be performed by a method of changing parts of polymer compounds by changing a temperature and a pH to bond the polymer compounds.

### (Physical Gel)

The physical gel is obtained by crosslinking polymer compounds by a non-covalent bond, that is, an ionic bond or a hydrogen bond and is also referred to as a weakly bonded gel or a physically crosslinked gel. The physical gel has characteristics that, since the strength of the crosslinking bond is weak and the crosslinking bond is broken due to the molecular motion of the polymer compound by thermal energy, the three-dimensional network structure of the gel composition is broken by thermal energy.

### (Physical Crosslinking)

Crosslinking in the physical gel, that is, physical crosslinking is performed, although not limited, for example, by freezing at a low temperature of −20° C. or lower. Preferably, the physical crosslinking is performed by freezing at a low temperature of −40 to −20° C. In the physical crosslinking by freezing, by adjusting a freezing temperature, freezing time, and the number of times of freezing, physical properties such as elasticity and viscosity of the hydrogel composition 10 can be changed.

Incidentally, the physical crosslinking may be performed by a repeated freeze-thaw method. In the repeated freeze-thaw method, by adjusting a freezing temperature, a thawing temperature, a freezing time, a thawing time, and the number of times of repeating freezing and thawing, physical properties such as elasticity and viscosity of the hydrogel composition 10 can be changed.

Incidentally, classification of the chemical gel and the physical gel described above is conveniently made for the purpose of describing the chemical crosslinking and the physical crosslinking, and it is not intended to classify the hydrogel composition 10 into any one of the chemical gel and the physical gel.

### (Crosslink Region 14 of Hydrogel Composition 10)

In the present invention, the crosslink region 14 of the hydrogel composition 10 can be configured to have only a physically crosslinked structure by a hydrogen bond of the hydroxyl group 5 and not to have a chemically crosslinked structure.

FIG. 3 is a schematic view illustrating a part of the crosslink region 14 by a hydrogen bond of the hydrogel composition 10 of the present invention. The basic skeleton 2 that is a linear alkyl chain of the polyvinyl alcohol 1 in FIG. 3 corresponds to the polymer chain 12 in the hydrogel composition 10 of FIG. 2. Further, as illustrated in FIG. 3, in the present invention, the crosslink region 14 of the hydrogel composition 10 is physically crosslinked by a hydrogen bond of the hydroxyl group 5 that is the functional group 3 of the polyvinyl alcohol 1.

That is, in the hydrogel composition 10, the resin of the polyvinyl alcohol 1 has a three-dimensional network structure. In addition, the hydrogel composition 10 has the low-molecular-weight solvent 18, which is confined to lose fluidity, inside the three-dimensional network structure of the resin of the polyvinyl alcohol 1.

### [Physical Properties of Hydrogel Composition 10]

The hydrogel composition 10 provides various physical properties depending on a difference in the degree of polymerization and the degree of saponification of the polyvinyl alcohol 1, the content of the solvent 18 contained in the hydrogel composition 10, and the like. Therefore, by using the hydrogel composition 10, various tissue models can be manufactured. Herein, the degree of polymerization and the degree of saponification of the polyvinyl alcohol 1 and the content of the solvent 18 in the hydrogel composition 10 which are some of parameters determining physical properties of the hydrogel composition 10 will be described.

Incidentally, “the content of the solvent 18 in the hydrogel composition 10” corresponds to “the solvent content” in the present application, and in the following description, is referred to “the solvent content in the hydrogel composition 10” or simply referred to as “the solvent content.”

### (Degree of Polymerization of Polyvinyl Alcohol 1)

The degree of polymerization of the polyvinyl alcohol 1 corresponds to the length of the basic skeleton 2 of the polyvinyl alcohol 1, and as the degree of polymerization increases, the basic skeleton 2 of the polyvinyl alcohol 1 is lengthened and the molecular weight of the polyvinyl alcohol 1 is increased. The degree of polymerization of the polyvinyl alcohol 1 is determined by the degree of polymerization of polyvinyl acetate in the polymerization process. The degree of polymerization of the polyvinyl alcohol 1 is calculated, for example, by measuring a molecular weight by a liquid chromatography method such as gel permeation chromatography.

Further, the degree of polymerization (DP) of the polyvinyl alcohol 1 is calculated by the following mathematical formula (2) in the case of using a variable m and a variable n illustrated in FIG. 1.

DP=m+n (2)

As the degree of polymerization of the polyvinyl alcohol 1 increases, physical properties such as viscosity, film strength, and water resistance of the hydrogel composition 10 are increased. In addition, as the degree of polymerization of the polyvinyl alcohol 1 increases, physical properties such as viscosity stability of the hydrogel composition 10 at a low temperature are decreased. Further, as the degree of polymerization of the polyvinyl alcohol 1 increases, the molecular weight and the intermolecular force of the polyvinyl alcohol 1 are increased, and thus the water solubility of the polyvinyl alcohol 1 is decreased and the viscosity thereof is increased.

### (Degree of Saponification of Polyvinyl Alcohol 1)

The degree of saponification of the polyvinyl alcohol 1 corresponds to a ratio (RH) of the hydroxyl group 5 in the functional group 3 of the polyvinyl alcohol 1, and as the degree of saponification increases, the amount of the hydroxyl group 5 of the polyvinyl alcohol 1 is increased. The degree of saponification of the polyvinyl alcohol 1 is determined by the amount of hydroxyl groups 5 in the polyvinyl alcohol 1 substituted for acetate groups 4 in the saponification process. The degree of saponification of the polyvinyl alcohol 1 is calculated, for example, by quantitating the amount of the acetate groups 4 of the polyvinyl alcohol 1 by using sodium hydroxide. Specifically, the degree of saponification (DS) is calculated as a numerical value of mol % unit by the following mathematical formula (3) by converting the quantitated amount of the acetate groups 4 into a molar percentage unit, that is, mol % unit as the ratio (RA) of the acetate groups 4 in the functional group 3 of the polyvinyl alcohol 1.

DS=RH=1−RA (3)

Further, the degree of saponification (DS) of the polyvinyl alcohol 1 is calculated as a numerical value of mol % unit by the following mathematical formula (4) in the case of using the variable m and the variable n illustrated in FIG. 1.

DS={n/(m+n)}×100 (4)

As the degree of saponification of the polyvinyl alcohol 1 increases, physical properties such as viscosity, film strength, and water resistance of the hydrogel composition 10 are increased. In addition, as the degree of saponification of the polyvinyl alcohol 1 increases, physical properties such as viscosity stability of the hydrogel composition 10 in a low temperature are decreased. Further, as the degree of saponification of the polyvinyl alcohol 1 increases, the polyvinyl alcohol 1 is easily crystallized, and thus the water solubility of the polyvinyl alcohol 1 is decreased and the viscosity thereof is increased.

### (Solvent Content of Hydrogel Composition 10)

The solvent content of the hydrogel composition 10 is calculated in wt % unit as the ratio of the weight of the solvent 18 to the weight of the hydrogel composition 10. As the solvent content increase, the wettability of the hydrogel composition 10 is increased, but the elasticity is decreased, and thus solid property and shape retaining property are deceased.

### [Model of Body Soft Tissue]

Next, the tissue model using the hydrogel composition 10 of the present invention will be described. The tissue model of the present invention can be manufactured as a model of a body soft tissue that particularly has physical properties such as viscosity, elasticity, and water retention property similar to physical properties of an actual body soft tissue by using the hydrogel composition 10.

Incidentally, in the following description, “the body soft tissue” means a tissue excluding a hard tissue of living body such as bone, tooth, or cartilage unless specified otherwise. The body soft tissue is not limited, but for example, includes a vascularized tissue, an oral soft tissue such as oral mucosa, a nasal soft tissue such as nasal mucosa, an aural soft tissue such as aural mucosa, a visceral tissue of brain, heart, liver, pancreas, spleen, kidney, bladder, lung, stomach, small intestine, large intestine, uterus, esophagus, or the like, a dermal tissue, a muscle tissue, an eyeball tissue, and the like. In addition, “the body hard tissue” means a hard tissue of living body such as bone, tooth, or cartilage.

### (Degree of Saponification and Degree of Polymerization of Polyvinyl Alcohol 1)

In the case of manufacturing a model of a body soft tissue as the tissue model of the present invention, polyvinyl alcohol 1 having a degree of saponification of 85 to 98 mol % is used. In a case where the degree of saponification of the polyvinyl alcohol 1 is less than 85 mol %, the elasticity of the model of the body soft tissue is decreased more than the elasticity of an actual body soft tissue by a decrease in film strength of the resin of the polyvinyl alcohol 1. In addition, in a case where the degree of polymerization of the polyvinyl alcohol 1 is more than 98 mol %, the viscosity of the model of the body soft tissue is increased more than the viscosity of an actual body soft tissue by an increase in viscosity of the resin of the polyvinyl alcohol 1. Further, the stationary surface friction coefficient of the model of the body soft tissue is increased more than the stationary surface friction coefficient of an actual body soft tissue by an increase in viscosity of the hydrogel composition 10. Therefore, in a case where the degree of saponification of the polyvinyl alcohol 1 is less than 85 mol % or more than 98 mol %, sensuality such as tactile sensation of the model of the body soft tissue is decreased.

Further, in the case of manufacturing a model of a body soft tissue, polyvinyl alcohol 1 having a degree of polymerization of 1000 to 2000 is used. In a case where the degree of polymerization of the polyvinyl alcohol 1 is less than 1000, the elasticity of the model of the body soft tissue is decreased more than the elasticity of an actual body soft tissue by a decrease in film strength of the hydrogel composition 10. In addition, in a case where the degree of polymerization of the polyvinyl alcohol 1 is more than 2000, the viscosity of the model of the body soft tissue is increased more than the viscosity of an actual body soft tissue by an increase in viscosity of the hydrogel composition 10. Further, the stationary surface friction coefficient of the model of the body soft tissue is increased more than the stationary surface friction coefficient of an actual body soft tissue by an increase in viscosity of the hydrogel composition 10. Therefore, in a case where the degree of polymerization of the polyvinyl alcohol 1 is less than 1000 or more than 2000, sensuality such as tactile sensation of the model of the body soft tissue is decreased.

Further, in the case of manufacturing a model of a body soft tissue, a polyvinyl alcohol resin can be produced by using only one kind of the polyvinyl alcohol 1 having a degree of saponification of 85 to 98 mol % and a degree of polymerization of 1000 to 2000. In addition, a polyvinyl alcohol resin having an average value of a degree of saponification of 85 to 98 mol % and an average value of a degree of polymerization of 1000 to 2000 can also be produced by using two or more kinds of the polyvinyl alcohol 1 having a different degree of saponification or degree of polymerization. Incidentally, in the present application, the term “average degree of saponification” corresponds to both “the degree of saponification” in the case of using only one kind of the polyvinyl alcohol 1 and “the average value of the degree of saponification” in the case of using two or more kinds of the polyvinyl alcohol 1. Further, in the present application, the term “average degree of polymerization” corresponds to both “the degree of polymerization” in the case of using only one kind of the polyvinyl alcohol 1 and “the average value of the degree of polymerization” in the case of using two or more kinds of the polyvinyl alcohol 1.

A preferable hydrogel composition 10 of the present invention contains a polyvinyl alcohol resin which is formed from two kinds of the polyvinyl alcohol 1 in which at least a degree of saponification is different and has an average degree of saponification of 85 to 98 mol % and an average degree of polymerization of 1000 to 2000. The above-described two kinds of the polyvinyl alcohol 1 includes a first polyvinyl alcohol and a second polyvinyl alcohol in which at least a degree of saponification is different from that of the first polyvinyl alcohol. Incidentally, the degree of polymerization of the second polyvinyl alcohol and the degree of polymerization of the first polyvinyl alcohol may be the same as or different from each other.

The ratio of an increase in viscosity and strength to an increase in degree of saponification is smaller than the ratio of an increase in viscosity and strength to an increase in degree of polymerization in the hydrogel composition 10 of the polyvinyl alcohol 1. Therefore, by adjusting the degree of saponification, the viscosity and the strength of the hydrogel composition 10 can be adjusted to a desired viscosity and a desired strength with a high degree of accuracy.

In a preferable hydrogel composition 10 of the present invention, by adjusting the average degree of saponification by using two kinds of the polyvinyl alcohol 1, the viscosity and the strength of the hydrogel composition 10 can be adjusted to a desired viscosity and a desired strength with ease and with a high degree of accuracy. Therefore, in the present invention, by using the above-described two kinds of the polyvinyl alcohol 1, as compared to the case of using one kind of the polyvinyl alcohol 1 singly, the hydrogel composition 10 having physical properties similar to those of an actual body soft tissue can be easily provided.

In the case of providing the hydrogel composition 10 of the present invention by using two kinds of the polyvinyl alcohol 1, as the first polyvinyl alcohol, preferably, polyvinyl alcohol 1 having a degree of saponification of 97 mol % or more and a degree of polymerization of 500 to 3000. As a more preferable first polyvinyl alcohol, polyvinyl alcohol 1 having a degree of saponification of 99 mol % or more and a degree of polymerization of 500 to 2000 is used.

The first polyvinyl alcohol can be produced by the method for producing the polyvinyl alcohol 1 described above. In addition, as the first polyvinyl alcohol, commercially available industrial products can be used. The first polyvinyl alcohol having a degree of saponification of 97 mol % or more and a degree of polymerization of 500 to 3000 is commercially available as a completely saponified industrial product. For example, the first polyvinyl alcohol is commercially available as industrial products such as JF-05, JF-10, JF-17, JF-20, V, VO, and VC-10 (trade names) from JAPAN VAM & POVAL CO., LTD. Further, the first polyvinyl alcohol is, for example, commercially available as industrial products such as PVA-105, PVA-110, PVA-117, PVA-117H, PVA-120, and PVA-124 (trade names) from Kuraray Co., Ltd. Among the above-described examples, the polyvinyl alcohol 1 having a degree of saponification of 99 mol % or more and a degree of polymerization having 500 to 2000 corresponds to industrial products such as JF-20, V, VO, and VC-10 of JAPAN VAM & POVAL CO., LTD. and PVA-117H of Kuraray Co., Ltd. (trade names).

Further, in the case of providing the hydrogel composition 10 of the present invention by using two kinds of the polyvinyl alcohol 1, as the second polyvinyl alcohol, preferably, polyvinyl alcohol 1 having a degree of saponification of 70 to 90 mol % or more and a degree of polymerization of 500 to 3000 is used. As a more preferable second polyvinyl alcohol, polyvinyl alcohol 1 having a degree of saponification of 86 to 90 mol % or more and a degree of polymerization of 500 to 2000 is used.

The second polyvinyl alcohol can be produced by the method for producing the polyvinyl alcohol 1 described above. In addition, as the second polyvinyl alcohol, commercially available industrial products can be used. The second polyvinyl alcohol having a degree of saponification of 70 to 90 mol % or more and a degree of polymerization of 500 to 3000 is commercially available as a partially saponified industrial product. For example, the second polyvinyl alcohol is commercially available as industrial products such as JP-05, JP-10, JP-15, JP-20, JP-24, VP-18, and VP-20 (trade names) from JAPAN VAM & POVAL CO., LTD. Further, the second polyvinyl alcohol is, for example, commercially available as industrial products such as PVA-205, PVA-210, PVA-217, PVA-220, and PVA-224 (trade names) from Kuraray Co., Ltd. Among the above-described examples, the polyvinyl alcohol 1 having a degree of saponification of 86 to 90 mol % or more and a degree of polymerization of 500 to 2000 corresponds to industrial products such as JP-5, JP-10, JP-15, JP-20, VP-18, and VP-20 of JAPAN VAM & POVAL CO., LTD. and PVA-205, PVA-210, PVA-217, and PVA-220 of Kuraray Co., Ltd. (trade names).

Incidentally, as the first polyvinyl alcohol and the second polyvinyl alcohol, any dried products of powdery or granular dried products can also be used. However, in the case of taking solubility and a degree of purifying of the dried product into consideration, the first polyvinyl alcohol and the second polyvinyl alcohol are preferably powdery dried products.

### (Weight Ratios of First Polyvinyl Alcohol and Second Polyvinyl Alcohol)

Further, the weight ratios of the first polyvinyl alcohol and the second polyvinyl alcohol in the polyvinyl alcohol resin are adjusted such that the average degree of saponification of the polyvinyl alcohol resin becomes 85 to 98 mol % and the average degree of polymerization thereof becomes 1000 to 2000. For example, in the case of manufacturing a model of a body soft tissue as a tissue model, the weight ratios of the first polyvinyl alcohol and the second polyvinyl alcohol are adjusted such that the weight ratio of the first polyvinyl alcohol becomes 99 to parts by weight and the weight ratio of the second polyvinyl alcohol becomes 1 to 30 parts by weight.

In a case where the weight ratio of the second polyvinyl alcohol in the polyvinyl alcohol resin is less than 1 part by weight, the elasticity of the model of the body soft tissue is increased more than the elasticity of an actual body soft tissue by an increase in elasticity of the hydrogel composition 10. Further, the normal force of the surface of the model of the body soft tissue is increased more than the normal force of the surface of an actual body soft tissue by an increase in elasticity of the hydrogel composition 10.

Further, in a case where the weight ratio of the second polyvinyl alcohol in the polyvinyl alcohol resin is less than 1 part by weight, the viscosity of the model of the body soft tissue is decreased more than the viscosity of an actual body soft tissue by a decrease in viscosity of the hydrogel composition 10. Further, the stationary surface friction coefficient of the model of the body soft tissue is smaller than the stationary surface friction coefficient of an actual body soft tissue by a decrease in viscosity of the hydrogel composition 10.

Conversely, in a case where the weight ratio of the second polyvinyl alcohol in the polyvinyl alcohol resin is more than 30 parts by weight, the elasticity of the model of the body soft tissue is decreased more than the elasticity of an actual body soft tissue by a decrease in elasticity of the hydrogel composition 10. Further, the normal force of the surface of the model of the body soft tissue is decreased more than the normal force of the surface of an actual body soft tissue by a decrease in elasticity of the hydrogel composition 10.

Further, in a case where the weight ratio of the second polyvinyl alcohol in the polyvinyl alcohol resin is more than 30 parts by weight, the viscosity of the model of the body soft tissue is increased more than the viscosity of an actual body soft tissue by an increase in viscosity of the hydrogel composition 10. Further, the stationary surface friction coefficient of the model of the body soft tissue is increased more than the stationary surface friction coefficient of an actual body soft tissue by an increase in viscosity of the hydrogel composition 10.

Therefore, in a case where the weight ratio of the second polyvinyl alcohol in the polyvinyl alcohol resin is less than 1 part by weight or more than 30 parts by weight, sensuality such as tactile sensation of the model of the body soft tissue is decreased.

Particularly, in the case of manufacturing a model of a body soft tissue including a wet mucosa such as oral soft tissue, nasal soft tissue, aural soft tissue, or an eyeball tissue as the model of the body soft tissue, in order to express properties such as wettability peculiar to the wet mucosa, it is necessary to secure sensuality such as tactile sensation of the model of the body soft tissue. Therefore, in the case of manufacturing a model or the like of a body soft tissue including a wet mucosa, in order to decrease the viscosity in the model of the body soft tissue, the upper limit of the weight ratio of the second polyvinyl alcohol in the polyvinyl alcohol resin can be set to be smaller than 30 parts by weight. In the case of manufacturing a model or the like of a body soft tissue including a wet mucosa, for example, the weight ratio of the first polyvinyl alcohol can be set to 99 to 80 parts by weight and the weight ratio of the second polyvinyl alcohol can be set to 1 to 20 parts by weight.

### (Solvent Content of Hydrogel Composition 10)

In the tissue model, the solvent content of the hydrogel composition 10 is adjusted such that physical properties such as elasticity similar to those of an actual soft tissue can be maintained and swelling property, solid property, and shape retaining property similar to those of an actual soft tissue can be provided. For example, the solvent content of the hydrogel composition 10 can be set, for example, to 70 to 95 wt %. In the case of manufacturing a model of a body soft tissue as the tissue model, a preferable solvent content of the hydrogel composition 10 is 70 to 90 wt % and a further preferable solvent content of the hydrogel composition 10 is 70 to 80 wt %.

### (Specific Example of Model of Body Soft Tissue)

Hereinafter, the average degree of saponification and the average degree of polymerization in the polyvinyl alcohol resin, the weight ratios of the first polyvinyl alcohol and the second polyvinyl alcohol, and the solvent content in the hydrogel composition 10 which are preferable for manufacturing the model of the body soft tissue are specifically exemplified.

For example, in the case of the body soft tissue being a blood vessel, a preferable average degree of saponification of the polyvinyl alcohol resin is 85 to 98 mol %. In addition, a preferable average degree of polymerization of the polyvinyl alcohol resin is 1000 to 2000. Further, a preferable weight ratio of the first polyvinyl alcohol in the polyvinyl alcohol resin is 99 to 70 parts by weight and a preferable weight ratio of the second polyvinyl alcohol in the polyvinyl alcohol resin is 1 to 30 parts by weight. Further, a preferable solvent content in the hydrogel composition 10 is 70 to 95 wt %.

For example, in the case of the body soft tissue being an oral soft tissue, a preferable average degree of saponification of the polyvinyl alcohol resin is 90 to 95 mol %. In addition, a preferable average degree of polymerization of the polyvinyl alcohol resin is 1000 to 2000. Further, a preferable weight ratio of the first polyvinyl alcohol in the polyvinyl alcohol resin is 99 to 70 parts by weight and a preferable weight ratio of the second polyvinyl alcohol in the polyvinyl alcohol resin is 1 to 30 parts by weight. Further, a preferable solvent content in the hydrogel composition 10 is 70 to 95 wt %.

For example, in the case of the body soft tissue being a nasal soft tissue, a preferable average degree of saponification of the polyvinyl alcohol resin is 90 to 98 mol %. In addition, a preferable average degree of polymerization of the polyvinyl alcohol resin is 1000 to 2000. Further, a preferable weight ratio of the first polyvinyl alcohol in the polyvinyl alcohol resin is 99 to 80 parts by weight and a preferable weight ratio of the second polyvinyl alcohol in the polyvinyl alcohol resin is 1 to 20 parts by weight. Further, a preferable solvent content in the hydrogel composition 10 is 70 to 95 wt %.

For example, in the case of the body soft tissue being an aural soft tissue, a preferable average degree of saponification of the polyvinyl alcohol resin is 90 to 98 mol %. In addition, a preferable average degree of polymerization of the polyvinyl alcohol resin is 1000 to 2000. Further, a preferable weight ratio of the first polyvinyl alcohol in the polyvinyl alcohol resin is 99 to 80 parts by weight and a preferable weight ratio of the second polyvinyl alcohol in the polyvinyl alcohol resin is 1 to 20 parts by weight. Further, a preferable solvent content in the hydrogel composition 10 is 70 to 95 wt %.

For example, in the case of the body soft tissue being a visceral tissue of stomach, intestine, liver, or the like, a preferable average degree of saponification of the polyvinyl alcohol resin is 85 to 98 mol %. In addition, a preferable average degree of polymerization of the polyvinyl alcohol resin is 1000 to 2000. Further, a preferable weight ratio of the first polyvinyl alcohol in the polyvinyl alcohol resin is 99 to 70 parts by weight and a preferable weight ratio of the second polyvinyl alcohol in the polyvinyl alcohol resin is 1 to 30 parts by weight. Further, a preferable solvent content in the hydrogel composition 10 is 70 to 95 wt %.

For example, in the case of the body soft tissue being a dermal tissue, a preferable average degree of saponification of the polyvinyl alcohol resin is 95 to 98 mol %. In addition, a preferable average degree of polymerization of the polyvinyl alcohol resin is 1000 to 2000. Further, a preferable weight ratio of the first polyvinyl alcohol in the polyvinyl alcohol resin is 99 to 80 parts by weight and a preferable weight ratio of the second polyvinyl alcohol in the polyvinyl alcohol resin is 1 to 20 parts by weight. Further, a preferable solvent content in the hydrogel composition 10 is 70 to 95 wt %.

As described above, according to the hydrogel composition 10 of the present invention, it is possible to provide a tissue model capable of making physical properties such as viscosity or elasticity similar to physical properties of an actual soft tissue. Particularly, in the case of manufacturing a model of a body soft tissue as the tissue model by using the hydrogel composition 10 of the present invention, it is possible to provide a tissue model suitable for detachment of a body soft tissue or surgery practice of incision or the like.

### [Additives to Hydrogel Composition 10]

### (Gelling Agent)

Incidentally, to the hydrogel composition 10 of the present invention, as necessary, a small amount of a gelling agent can be added as an adjuvant for physical crosslinking by a hydrogen bond. The gelling agent is not limited, but for example, borates such as sodium tetraborate are used. In addition, the amount of the gelling agent added to the hydrogel composition 10 is not limited, but for example, can be set to 5 wt % or less.

### (Antiseptic)

Further, to the hydrogel composition 10 of the present invention, as necessary, a small amount of an antiseptic for providing storage stability can be added. The antiseptic is not limited, but for example, dehydroacetate, sorbate, benzoate, pentachlorophenol sodium, 2-pyridinethiol-1-oxide sodium, 2,4-dimethyl-6-acetoxy-m-dioxane, 1,2-benzthiazolin-3-one, or the like is added. The amount of the antiseptic added to the hydrogel composition 10 is not limited, but for example, can be set to 1 wt % or less.

### (Colorant Such as Dye or Pigment)

Further, to the hydrogel composition 10 of the present invention, as necessary, a small amount of a colorant can be added in order to replicate color of an actual soft tissue. The amount of the colorant added to the hydrogel composition 10 is not limited, but for example, can be set to 1 wt % or less. The colorant is not limited, but for example, a dye, a pigment, or the like is used.

### (Dye)

As the dye, a black dye, a magenta dye, a cyan dye, and a yellow dye are used. Hereinafter, specific examples of the black dye, the magenta dye, the cyan dye, and the yellow dye which can be used in the hydrogel composition 10 of the present invention are listed and illustrated, but are not intended to be limited to the following dyes.

As the black dye, for example, MS BLACK VPC manufactured by Mitsui Chemicals, Inc., AIZEN SOT BLACK-1 and AIZEN SOT BLACK-5 manufactured by Hodogaya Chemical Co., Ltd., KAYASET BLACK A-N manufactured by Nippon Kayaku Co., Ltd., DAIWA BLACK MSC manufactured by DaiwaKasei Co., Ltd., HSB-202 manufactured by Mitsubishi Chemical Corporation, NEPTUNE BLACK X60 and NEOPEN BLACK X58 manufactured by BASF Japan Ltd., Oleosol Fast BLACK RL manufactured by TAOKA CHEMICAL COMPANY, LIMITED, Chuo BLACK80 and Chuo BLACK80-15 manufactured by CHUO SYNTHETIC CHEMICAL CO., LTD., and the like are used.

As the magenta dye, for example, MS Magenta VP, MS Magenta HM-1450, and MS Magenta Hso-147 manufactured by Mitsui Chemicals, Inc., AIZEN SOT Red-1, AIZEN SOT Red-2, AIZEN SOT Red-3, AIZEN SOT Pink-1, and SPIRON Red GEHSPECIAL manufactured by Hodogaya Chemical Co., Ltd., KAYASET Red B, KAYASET Red 130, and KAYASET Red 802 manufactured by Nippon Kayaku Co., Ltd., PHLOXIN, ROSE BENGAL, and ACID Red manufactured by DaiwaKasei Co., Ltd., HSR-31 and DIARESIN Red K manufactured by Mitsubishi Chemical Corporation, Oil Red manufactured by BASF Japan Ltd., Oil Pink 330 manufactured by CHUG SYNTHETIC CHEMICAL CO., LTD., and the like are used.

As the cyan dye, for example, MS Cyan HM-1238, MS Cyan HSo-16, Cyan Hso-144, and MS Cyan VPG manufactured by Mitsui Chemicals, Inc., AIZEN SOT Blue-4 manufactured by Hodogaya Chemical Co., Ltd., KAYASET Blue Fr, KAYASET Blue N, KAYASET Blue 814, Turq. Blue GL-5 200, and Light Blue BGL-5 200 manufactured by Nippon Kayaku Co., Ltd., DAIWA Blue 7000 and Oleosol Fast Blue GL manufactured by DaiwaKasei Co., Ltd., DIARESIN Blue P manufactured by Mitsubishi Chemical Corporation, SUDAN Blue 670, NEOPEN Blue 808, and ZAPON Blue 806 manufactured by BASF Japan Ltd., and the like are used.

As the yellow dye, for example, MS Yellow HSm-41, Yellow KX-7, and Yellow EX-27 manufactured by Mitsui Chemicals, Inc., AIZEN SOT Yellow-1, AIZEN SOT YelloW-3, and AIZEN SOT Yellow-6 manufactured by Hodogaya Chemical Co., Ltd., KAYASET Yellow SF-G, KAYASET Yellow 2G, KAYASET Yellow A-G, and KAYASET Yellow E-G manufactured by Nippon Kayaku Co., Ltd., DAIWA Yellow 330HB manufactured by DaiwaKasei Co., Ltd., HSY-68 manufactured by Mitsubishi Chemical Corporation, SUDAN Yellow 146 and NEOPEN Yellow 075 manufactured by BASF Japan Ltd., Oil Yellow 129 manufactured by CHUG SYNTHETIC CHEMICAL CO., LTD., and the like are used.

### (Pigment)

Further, as the pigment, an organic pigment or an inorganic pigment can be used. For example, as the organic pigment or the inorganic pigment, azo pigments such as azolake, an insoluble azo pigment, a condensed azo pigment, and a chelate azo pigment can be used. In addition, as the organic pigment or the inorganic pigment, polycyclic pigments such as a phthalocyanine pigment, a perylene pigment, an anthraquinone pigment, a quinacridone pigment, a dioxazine pigment, a thioindigo pigment, an isoindolinone pigment, and a quinophthalone pigment can be used. Furthermore, as the organic pigment or the inorganic pigment, color pigments such as a red or magenta pigment, a blue or cyan pigment, a green pigment, a yellow pigment, and a black pigment can be used. Hereinafter, specific examples of pigments of numbers described in the color index which can be used in the hydrogel composition 10 of the present invention are listed and illustrated, but are not intended to be limited to the following pigments.

As the red or magenta pigment, for example, Pigment Red 3, Pigment Red 5, Pigment Red 19, Pigment Red 22, Pigment Red 31, Pigment Red 38, Pigment Red 43, Pigment Red 48:1, Pigment Red 48:2, Pigment Red 48:3, Pigment Red 48:4, Pigment Red 48:5, Pigment Red 49:1, Pigment Red 53:1, Pigment Red 57:1, Pigment Red 57:2, Pigment Red 58:4, Pigment Red 63:1, Pigment Red 81, Pigment Red 81:1, Pigment Red 81:2, Pigment Red 81:3, Pigment Red 81:4, Pigment Red 88, Pigment Red 104, Pigment Red 108, Pigment Red 112, Pigment Red 122, Pigment Red 123, Pigment Red 144, Pigment Red 146, Pigment Red 149, Pigment Red 166, Pigment Red 168, Pigment Red 169, Pigment Red 170, Pigment Red 177, Pigment Red 178, Pigment Red 179, Pigment Red 184, Pigment Red 185, Pigment Red 208, Pigment Red 216, Pigment Red 226, Pigment Red 257, Pigment Violet 3, Pigment Violet 19, Pigment Violet 23, Pigment Violet 29, Pigment Violet 30, Pigment Violet 37, Pigment Violet 50, Pigment Violet 88, Pigment Orange 13, Pigment Orange 16, Pigment Orange 20, Pigment Orange 36, and the like are used.

As the blue or cyan pigment, for example, Pigment Blue 1, Pigment Blue 15, Pigment Blue 15:1, Pigment Blue 15:2, Pigment Blue 15:3, Pigment Blue 15:4, Pigment Blue 15:6, Pigment Blue 16, Pigment Blue 17-1, Pigment Blue 22, Pigment Blue 27, Pigment Blue 28, Pigment Blue 29, Pigment Blue 36, Pigment Blue 60, and the like are used.

As the green pigment, for example, Pigment Green 7, Pigment Green 26, Pigment Green 36, Pigment Green 50, and the like are used.

As the yellow pigment, for example, Pigment Yellow 1, Pigment Yellow 3, Pigment Yellow 12, Pigment Yellow 13, Pigment Yellow 14, Pigment Yellow 17, Pigment Yellow 34, Pigment Yellow 35, Pigment Yellow 37, Pigment Yellow 55, Pigment Yellow 74, Pigment Yellow 81, Pigment Yellow 83, Pigment Yellow 93, Pigment Yellow 94, Pigment Yellow 95, Pigment Yellow 97, Pigment Yellow 108, Pigment Yellow 109, Pigment Yellow 110, Pigment Yellow 137, Pigment Yellow 138, Pigment Yellow 139, Pigment Yellow 153, Pigment Yellow 154, Pigment Yellow 155, Pigment Yellow 157, Pigment Yellow 166, Pigment Yellow 167, Pigment Yellow 168, Pigment Yellow 180, Pigment Yellow 185, Pigment Yellow 193, and the like are used.

As the black pigment, for example, Pigment Black 7, Pigment Black 26, Pigment Black 28, and the like are used.

Further, as the pigment, commercially available industrial products can be used. Hereinafter, specific examples of commercially available pigments are listed and illustrated, but are neither intended to be limited to the above-described pigments nor intended to be limited to industrial products manufactured by the following manufacturers.

Examples of pigments as industrial products include Chromo Fine Yellow 2080, Chromo Fine Yellow 5900, Chromo Fine Yellow 5930, Chromo Fine Yellow AF-1300, Chromo Fine Yellow 2700L, Chromo Fine Orange 3700L, Chromo Fine Orange 6730, Chromo Fine Scarlet 6750, Chromo Fine Magenta 6880, Chromo Fine Magenta 6886, Chromo Fine Magenta 6891N, Chromo Fine Magenta 6790, Chromo Fine Magenta 6887, Chromo Fine Violet RE, Chromo Fine Red 6820, Chromo Fine Red 6820, Chromo Fine Red 6830, Chromo Fine Blue HS-3, Chromo Fine Blue 5187, Chromo Fine Blue 5108, Chromo Fine Blue 5197, Chromo Fine Blue 5085N, Chromo Fine Blue SR-5020, Chromo Fine Blue 5026, Chromo Fine Blue 5050, Chromo Fine Blue 4920, Chromo Fine Blue 4927, Chromo Fine Blue 4937, Chromo Fine Blue 4824, Chromo Fine Blue 4933GN-EP, Chromo Fine Blue 4940, Chromo Fine Blue 4973, Chromo Fine Blue 5205, Chromo Fine Blue 5208, Chromo Fine Blue 5214, Chromo Fine Blue 5221, Chromo Fine Blue 5000P, Chromo Fine Green 2GN, Chromo Fine Green 2GO, Chromo Fine Green 2G-550D, Chromo Fine Green 5310, Chromo Fine Green 5370, Chromo Fine Green 6830, Chromo Fine Black A-1103, Seika Fast Yellow 10tH, Seika Fast Yellow A-3, Seika Fast Yellow 2035, Seika Fast Yellow 2054, Seika Fast Yellow 2200, Seika Fast Yellow 2270, Seika Fast Yellow 2300, Seika Fast Yellow 2400(B), Seika Fast Yellow 2500, Seika Fast Yellow 2600, Seika Fast Yellow ZAY-260, Seika Fast Yellow 2700(B), Seika Fast Yellow 2770, Seika Fast Red 8040, Seika Fast Red C405(F), Seika Fast Red CA120, Seika Fast Red LR-116, Seika Fast Red 1531B, Seika Fast Red 8060R, Seika Fast Red 1547, Seika Fast Red ZAW-262, Seika Fast Red 1537B, Seika Fast Red GY, Seika Fast Red 4R-4016, Seika Fast Red 3820, Seika Fast Red 3891, Seika Fast Red ZA-215, Seika Fast Carmine 6B1476T-7, Seika Fast Carmine 1483LT, Seika Fast Carmine 3840, Seika Fast Carmine 3870, Seika Fast Bordeaux 10B-430, Seika Light Rose R40, Seika Light Violet B800, Seika Light Violet 7805, Seika Fast Maroon 460N, Seika Fast Orange 900, Seika Fast Orange 2900, Seika Light Blue C718, Seika Light Blue A612, Cyanine Blue 4933M, Cyanine Blue 4933GN-EP, Cyanine Blue 4940, and Cyanine Blue 4973 (trade names, all commercially available from Dainichiseika Color & Chemicals Mfg. Co., Ltd.).

Further, examples of pigments as industrial products include KET Yellow 401, KET Yellow 402, KET Yellow 403, KET Yellow 404, KET Yellow 405, KET Yellow 406, KET Yellow 416, KET Yellow 424, KET Orange 501, KET Red 301, KET Red 302, KET Red 303, KET Red 304, KET Red 305, KET Red 306, KET Red 307, KET Red 308, KET Red 309, KET Red 310, KET Red 336, KET Red 337, KET Red 338, KET Red 346, KET Blue 101, KET Blue 102, KET Blue 103, KET Blue 104, KET Blue 105, KET Blue 106, KET Blue 111, KET Blue 118, KET Blue 124, and KET Green 201 (trade names, all commercially available from DIC Corporation).

Further, examples of pigments as industrial products include Colortex Yellow 301, Colortex Yellow 314, Colortex Yellow 315, Colortex Yellow 316, Colortex Yellow P-624, Colortex Yellow U10GN, Colortex Yellow U3GN, Colortex Yellow UNN, Colortex Yellow UA-414, Colortex Yellow U263, Finecol Yellow T-13, Finecol Yellow T-05, Pigment Yellow 1705, Colortex Orange 202, Colortex Red 101, Colortex Red 103, Colortex Red 115, Colortex Red 116, Colortex Red D3B, Colortex Red P-625, Colortex Red 102, Colortex Red H-1024, Colortex Red 105C, Colortex Red UFN, Colortex Red UCN, Colortex Red UBN, Colortex Red U3BN, Colortex Red URN, Colortex Red UGN, Colortex Red UG276, Colortex Red U456, Colortex Red U457, Colortex Red 105C, Colortex Red USN, Colortex Maroon 601, Colortex Brown B610N, Colortex Violet 600, Pigment Red 122, Colortex Blue 516, Colortex Blue 517, Colortex Blue 518, Colortex Blue 519, Colortex Blue A818, Colortex Blue P-908, Colortex Blue 510, Colortex Green 402, Colortex Green 403, Colortex Black 702, and Colortex Black U905 (trade names, all commercially available from SANYO COLOR WORKS, Ltd.).

Further, examples of pigments as industrial products include LIONOL YELLOW 1405G, LIONOL BLUE FG7330, LIONOL BLUE FG7350, LIONOL BLUE FG7400G, LIONOL BLUE FG7405G, LIONOL BLUE ES, and LIONOL BLUE ESP-S (trade names, all commercially available from TOYO INK CO., LTD.).

Further, examples of pigments as industrial products include Carbon Black #2600, Carbon Black #2400, Carbon Black #2350, Carbon Black #2200, Carbon Black #1000, Carbon Black #990, Carbon Black #980, Carbon Black #970, Carbon Black #960, Carbon Black #950, Carbon Black #850, Carbon Black MCF88, Carbon Black #750, Carbon Black #650, Carbon Black MA600, Carbon Black MA7, Carbon Black MA8, Carbon Black MA11, Carbon Black MA100, Carbon Black MA100R, Carbon Black MA77, Carbon Black #52, Carbon Black #50, Carbon Black #47, Carbon Black #45, Carbon Black #45L, Carbon Black #40, Carbon Black #33, Carbon Black #32, Carbon Black #30, Carbon Black #25, Carbon Black #20, Carbon Black #10, Carbon Black #5, and Carbon Black #44 (trade names, all commercially available from Mitsubishi Chemical Corporation).

### (Water-Swellable Layered Clay Mineral)

Further, in the case of manufacturing, for example, a model of an organ such as heart as the tissue model, in order to make physical properties such as elasticity of the model of the organ similar to physical properties of an actual organ, a water-swellable layered clay mineral can be added to the hydrogel composition 10 of the present invention as necessary. The water-swellable layered clay mineral can be dispersed in the solvent 18 and is a clay mineral having a layered structure. The amount of the water-swellable layered clay mineral added to the hydrogel composition 10 is not limited, but for example, can be set to 1 to 5 wt %. As the water-swellable layered clay mineral, although not limited, for example, water swellable smectite such as water swellable Hectorite, water swellable montmorillonite, and water swellable saponite, and water swellable mica such as water swellable synthetic mica can be used. In addition, water swellable smectite and water swellable mica can be formed to be a clay mineral in which sodium ions are contained between clay mineral layers.

Incidentally, as the water-swellable layered clay mineral, only one kind of water-swellable layered clay mineral may be used singly or two or more kinds of water-swellable layered clay minerals may be used. In addition, the water-swellable layered clay mineral can be used as a synthetic product of two or more kinds of water-swellable layered clay minerals. Further, as the water-swellable layered clay mineral, commercially available industrial products can be used. As the water-swellable layered clay mineral that is an industrial product, although not limited, for example, synthetic Hectorite SWN, fluorinated Hectorite SWF, and the like manufactured by Katakura & Co-op Agri Corporation can be used.

When the water-swellable layered clay mineral is added to the hydrogel composition 10 of the present invention, physical properties such as elasticity of the model of the organ, that is, mechanical strength can be made similar to mechanical strength of an actual organ. In other words, when the water-swellable layered clay mineral is added to the hydrogel composition 10 of the present invention, sensuality such as tactile sensation of the model of the organ can be made similar to that of an actual organ. Therefore, when the water-swellable layered clay mineral is added to the hydrogel composition 10 of the present invention, for example, tactile sensation at the time of incision by a surgical scalpel or the like can be made similar to tactile sensation of an actual organ, and thus a model of an organ suitable for surgery practice can be provided.

### [Method for Producing Hydrogel Composition 10]

Next, an example of a method for producing the hydrogel composition 10 of the present invention will be described in detail.

The hydrogel composition 10 of the present invention can be produced by a method including: a step of mixing a first polyvinyl alcohol having a degree of saponification of 97% or more and a degree of polymerization of 500 to 2000 and a second polyvinyl alcohol having a degree of saponification of 86 to 90% and a degree of polymerization of 500 to 2000 in a first solvent; a step of heating the first solvent mixed with the first polyvinyl alcohol and the second polyvinyl alcohol at a first temperature to produce a solution in which the first polyvinyl alcohol and the second polyvinyl alcohol are dissolved in the first solvent; and a step of causing the solution to gel into a polyvinyl alcohol hydrogel containing a polyvinyl alcohol resin with a three-dimensional network structure having a plurality of polymer chains and a crosslink region bonding the plurality of polymer chains to each other and the first solvent which is confined in a network portion of the three-dimensional network structure to lose fluidity, by freezing the solvent at a second temperature.

Incidentally, in the following description, the above-described “first solvent” corresponds to the “solvent 18” or the “mixed solvent.” Further, in the following description, the above-described “first temperature” corresponds to a “heating temperature.” Furthermore, the above-described “second temperature” corresponds to a “freezing temperature.”

(Mixing of Mixture of Polyvinyl Alcohol 1 with Solvent 18)

The mixture of the polyvinyl alcohol 1 constituting the polyvinyl alcohol resin can be produced, for example, by mixing 99 to 70 parts by weight of powder of the first polyvinyl alcohol and 1 to 30 parts by weight of powder of the second polyvinyl alcohol. The mixture of the polyvinyl alcohol 1 is added to the solvent 18. The amount of the mixture of the polyvinyl alcohol 1 added to the solvent 18 can be set to an arbitrary amount in order to obtain preferable physical properties of the hydrogel composition 10. For example, 15 parts by weight of the mixture of the polyvinyl alcohol 1 may be added to 85 parts by weight of the solvent 18 or 17 parts by weight of the mixture of the polyvinyl alcohol 1 may be added to 83 parts by weight of the solvent 18. Further, the polyvinyl alcohol resin may be configured by only powder of one kind of the first polyvinyl alcohol. In the following description, the “mixture of the polyvinyl alcohol 1” also includes a composition formed from only powder of one kind of the first polyvinyl alcohol.

As the solvent 18 for production of the hydrogel composition 10 of the present invention, for example, a mixed solvent of water and dimethyl sulfoxide that is a mixed solvent which is excellent in solubility of the polyvinyl alcohol 1 and is not frozen at a low temperature is used. Incidentally, dimethyl sulfoxide is abbreviated as DMSO in the following description.

Further, the weight ratio of DMSO to water in the mixed solvent is not limited, but for example, can be set to 1 to 10. A preferable weight ratio of DMSO to water in production of the hydrogel composition 10 of the present invention is 1 to 5. A most preferable weight ratio of DMSO to water in production of the hydrogel composition 10 of the present invention is 4.

### (Production of Solution of Polyvinyl Alcohol 1)

The solvent 18 added with the mixture of the polyvinyl alcohol 1 is heated, the mixture of the polyvinyl alcohol 1 is dissolved in the solvent 18 while being stirred with a stirrer, and thus a solution of polyvinyl alcohol is produced. The heating temperature of the solvent 18 can be set, for example, to 60 to 120° C. Further, in the case of taking the degree of solubility or the like of the polyvinyl alcohol 1 into consideration, a preferable heating temperature of the solvent 18 is 100 to 120° C. Incidentally, in the case of taking the boiling point or the like of water in the solvent 18 into consideration, a most preferable heating temperature of the solvent 18 is 100° C.

Incidentally, the mixture of the polyvinyl alcohol 1 being dissolved in the solvent 18 may be performed in an opened state or in a sealed state, but taking prevention of mixing of impurities into consideration, is preferably performed in a sealed state. Further, the solution of the polyvinyl alcohol 1 may be produced by mixing a solution obtained by dissolving the first polyvinyl alcohol in the solvent 18 and a solution obtained by dissolving the second polyvinyl alcohol in the solvent 18.

### (Gelation of Solution of Polyvinyl Alcohol 1)

The solution of the polyvinyl alcohol 1 gels into a polyvinyl alcohol hydrogel by freezing. The freezing temperature for causing the solution to gel can be set, for example, to −20° C. or lower. Further, in the case of taking physical properties such as viscosity and elasticity into consideration, a preferable freezing temperature of the hydrogel composition 10 is −40 to −20° C. or lower and a most preferable freezing temperature is −30° C.

### [Method for Producing Dried Tissue Model]

Next, an example of a method for producing the dried tissue model of the present invention will be described in detail.

The dried tissue model of the present invention can be produced, for example, by a method including the steps of injecting a solution of the polyvinyl alcohol 1 into a mold for a tissue model, causing the solution of the polyvinyl alcohol 1 injected into the mold to gel so that a tissue model is produced, and taking out the produced tissue model of the mold and then drying the tissue model.

In the following description, the case of producing a model of a body soft tissue such as a vascularized tissue or an oral soft tissue as the tissue model will be considered.

### (Mold for Tissue Model)

The mold for the tissue model can be manufactured, for example, by cutting work, stereolithography, molding processing using a modeling device such as a 3D printer, or the like. A material for the mold for the tissue model is not particularly limited as long as it is a material with which the hydrogel composition 10 can be produced into a shape of a tissue, but for example, a material such as a silicone resin, quartz glass, a metal, gypsum, wax, or a synthetic resin can be used. For example, in the case of a metal such as brass, stainless steel, nickel titanium, or alumina as the material for the mold for the tissue, thermal conduction at the time of a freezing treatment can be improved and the freezing time can be decreased.

(Injection of Solution of Polyvinyl Alcohol 1 into Mold for Tissue Model)

In the method for producing the dried tissue model, the solution of the polyvinyl alcohol 1 is injected into the mold for the tissue model. For example, the solution of the polyvinyl alcohol 1 is injected into the mold for the tissue model under a pressurized condition of 150 to 160 kg/cm². By setting the pressurized condition at the time of injection to 150 to 160 kg/cm², it is possible to reduce air bubbles generated in the injected solution of the polyvinyl alcohol 1 and homogenize the injected solution of the polyvinyl alcohol 1.

Incidentally, the method for producing the solution of the polyvinyl alcohol 1 includes the same contents as the contents described in “Mixing of Mixture of Polyvinyl Alcohol 1 with Solvent 18” and “Production of Solution of Polyvinyl Alcohol 1” in the method for producing the hydrogel composition 10 described above, and thus the description thereof will be emitted.

(Gelation of Solution of Polyvinyl Alcohol 1 Injected into Mold for Tissue Model)

Next, the mold for the tissue model into which the solution of the polyvinyl alcohol 1 has been injected is subjected to a freezing treatment, so that the solution of the polyvinyl alcohol 1 inside the mold for the tissue model gels into a polyvinyl alcohol hydrogel. A freezing temperature for causing the solution of the polyvinyl alcohol 1 to gel can be set, for example, to −20° C. or lower. Further, in the case of taking physical properties such as viscosity and elasticity of the tissue model (the hydrogel composition 10) into consideration, a preferable freezing treatment temperature of the mold for the tissue model is −40 to −20° C. or lower and a most preferable freezing temperature is −30° C. Further, a preferable freezing treatment time for the mold for the tissue model is 24 hours or longer and a most preferable freezing treatment time is 24 hours. Further, the number of times of the freezing treatment of the mold for the tissue model may be plural times, also taking physical properties of the hydrogel composition 10 into consideration.

### (Drying of Manufactured Tissue Model)

The manufactured tissue model is taken out of the mold and subjected to a drying treatment, thereby completing a dried tissue model. The drying treatment may be performed by a drying apparatus such as an incubator or may be performed by natural drying. In the case of performing the drying treatment by a drying apparatus, the drying treatment is performed, for example, at a temperature of 40° C. for 72 hours or longer.

In the method for producing the dried tissue model of the present invention, before the tissue model is taken out of the mold, a water substitution treatment in which dimethyl sulfoxide ethanol contained in the hydrogel composition 10 is replaced by water may be performed. For example, in the water substitution treatment of the hydrogel composition 10, first, the mold for the tissue model in which the freezing treatment has been completed is immersed in a sufficient amount of ethanol for 120 minutes so that a treatment in which the dimethyl sulfoxide in the hydrogel composition 10 is replaced by ethanol is performed. Next, the mold for the tissue model is immersed in a sufficient amount of water for 24 hours so that a treatment in which the ethanol in the hydrogel composition 10 is replaced by water is performed.

Incidentally, the dried tissue model may be produced by a method other than the above-described method. For example, the dried tissue model may also be produced by a treatment other than the above-described treatment in which the solution of the polyvinyl alcohol 1 is injected into the mold for the tissue model. The dried tissue model may also be produced by compressively molding the solution of the polyvinyl alcohol 1 on the mold for the tissue model by using a modeling device such as a 3D printer that is an inkjet type material jetting modeling device. Further, the dried tissue model can also be produced by applying the solution of the polyvinyl alcohol 1 to the mold for the tissue model.

Further, the mold for the model of the soft tissue can partially include a model of a body hard tissue of bone, tooth, cartilage, or the like. The model of a body hard tissue of bone, tooth, cartilage, or the like can be manufactured, for example, from gypsum, wood, paper, a metal, or a synthetic resin such as an acrylic resin.

For example, a model of bone can be manufactured by adding sawdust and polyvinyl alcohol to an acrylic resin. In the model of bone, the hardness of bone can be replicated according to an amount of the acrylic resin used, and the tactile sensation of bone can be replicated according to a particle size of the sawdust. Further, in the model of bone, by adding polyvinyl alcohol, a close contact state between the bone and the body soft tissue inside the living body can be replicated.

Further, the model of the body soft tissue can manufactured as a part of a living body model such as an oral cavity model, a nasal cavity model, an aural cavity model, an eye model, a head model, a chest model, or an abdomen model.

For example, in the case of manufacturing an oral cavity model as the living body model, the oral cavity model can be manufactured by injecting the solution of the polyvinyl alcohol 1 into a mold for an oral cavity model including the above-described bone model, causing the solution of the polyvinyl alcohol 1 to gel, and then taking out the mold other than the model of the body hard tissue. Further, the oral cavity model can also be manufactured by compressively molding the solution of the polyvinyl alcohol 1 on the above-described bone model by using a modeling device such as a 3D printer and causing the solution of the polyvinyl alcohol 1 to gel into the hydrogel composition 10.

In the above-described oral cavity model, a close contact state between the bone model and the model of the oral tissue such as oral mucosa can be made similar to a close contact state between bone and an oral tissue inside a living body. Further, in the above-described oral cavity model, the model of the body soft tissue can be uniformly detached from the model of the body hard tissue by a surgical cutting instrument such as a surgeon's knife. Therefore, in the above-described oral cavity model, feeling similar to a detachment surgery of oral mucosa inside the living body can be replicated.

### EXAMPLES

The present invention will be described in detail by means of the following Examples, but the present invention is not limited to these Examples.

### [Example 1] (Preparation of Sample of Hydrogel Composition)

Only powder of the first polyvinyl alcohol was used as the powder of the polyvinyl alcohol constituting the polyvinyl alcohol resin. As the powder of the first polyvinyl alcohol, an industrial product manufactured by JAPAN VAM & POVAL CO., LTD., trade name: J-POVAL V, which is prepared to have a degree of saponification of 99.0 mol % or more and a degree of polymerization of 1700, was used.

15 parts by weight of the polyvinyl alcohol described above was mixed with 85 parts by weight of a mixed solvent of dimethyl sulfoxide and water. The obtained mixture was stirred for 2 hours under a temperature condition of 100° C. to dissolve the polyvinyl alcohol mixture in the mixed solvent, thereby producing a polyvinyl alcohol solution. As the mixed solvent of dimethyl sulfoxide and water, an industrial product manufactured by Toray Fine Chemicals Co., Ltd., which is prepared to have a weight ratio of dimethyl sulfoxide to water of 4, was used.

The produced polyvinyl alcohol solution was cooled to 40° C. under a thermoneutral environment. Thereafter, the polyvinyl alcohol solution was injected into a mold, and the mold into which the polyvinyl alcohol solution was injected was cooled for 24 hours under a temperature condition of −30° C., thereby preparing a sample of a hydrogel composition. As the mold, a rectangular stainless steel mold having a long side length of 50 mm, a short side length of 8 mm, and a thickness of 1 mm was used.

The mixed solvent of dimethyl sulfoxide and water described in Example 1 is an example of the first solvent.

### Example 2

### (Measurement of Tensile Stress of Hydrogel Composition)

The tensile stress of the sample of the hydrogel composition prepared by the method described in Example 1 was measured. In measurement of the tensile stress of the sample of the hydrogel composition, a small table-top testing machine, Model No. EZ-S, manufactured by SHIMADZU CORPORATION was used as a uniaxial tensile testing machine. Both ends at the long side of the sample of the hydrogel composition were held with grips of the uniaxial tensile testing machine to fix the sample of the hydrogel composition such that an initial distance between the grips would be 40 mm.

Next, the fixed sample of the hydrogel composition was pulled in both directions at a speed of 20 mm/min until the strain of the sample of the hydrogel composition would be 100%, and then the strain of the sample of the hydrogel composition was restored to 0%. That is, the fixed sample of the hydrogel composition was pulled in both directions until the distance between the grips would be two times the initial distance, and then the distance between the grips was restored to the initial distance. In Example 2, the fixed sample of the hydrogel composition was pulled until the distance therebetween would be 80 mm, and then the distance therebetween was restored to 40 mm.

In Example 2, taking hysteresis of the sample of the hydrogel composition into consideration, the above-described operation cycle was repeated three times, and the strain of the sample of the hydrogel composition and the tensile stress of the sample of the hydrogel composition in the third cycle were measured for every 0.05 seconds.

Values of tensile stress of the sample of the hydrogel composition with respect to strains of 25%, 50%, 75%, and 100% are shown in the following Table 1. In Table 1, a strain ΔL/L is represented by the ratio of elongation ΔL of the sample of the hydrogel composition to an initial distance L between the grips. That is, values of strains of “0.25,” “0.5,” “0.75,” and “1” in Table 1 respectively correspond to strains of 25%, 50%, 75%, and 100%. In addition, the tensile stress was represented by kilopascal (kPa) unit, and was calculated from measurement values of tensile stress corresponding to measurement values of strains best approximation to strains of 25%, 50%, 75%, and 100%. Based on Table 1, for example, the hydrogel composition can be configured such that the tensile stress with respect to a strain of 0.25 to 0.5 becomes 64.0 to 146.0 kPa.

TABLE 1

| Strain (ΔL/L) | 0.25 | 0.50 | 0.75 | 1.00 |
|---|---|---|---|---|
| Stress (kPa) | 64.0 | 146.0 | 274.1 | 447.5 |

Further, the Young's modulus that is an index for an elasticity in tension of the sample of the hydrogel composition was calculated from the results of the tensile test. As shown in FIG. 10 described later, in the sample of the hydrogel composition, when a value of strain is larger than 0.6, non-linearity between the strain and the tensile stress becomes significant. Therefore, regarding the sample of the hydrogel composition, the Young's modulus was calculated as a Young's modulus at a strain of 50% in which linearity of the Young's modulus is maintained. The Young's modulus of the sample of the hydrogel composition at a strain of 50% was 292 (kPa).

The Young's modulus of the hydrogel composition at a strain of 50% described in Example 2 corresponds to a “first Young's modulus.” In addition, the tensile stress of the hydrogel composition described in Example 2 corresponds to a “first stress” or “the stress of the hydrogel composition.”

### Example 3

### (Preparation of Sample of Dried Hydrogel)

Four samples of a hydrogel composition were prepared by the method described in Example 1. The prepared four samples of the hydrogel composition were disposed in an incubator. The mixed solvent of dimethyl sulfoxide and water contained in the sample of the hydrogel composition was vaporized inside the incubator for 168 hours. The internal temperature of the incubator was maintained to 40° C.

A dried product of the hydrogel composition obtained by vaporizing the mixed solvent of dimethyl sulfoxide and water, which is an example of the first solvent, from the hydrogel composition is referred to as a “dried hydrogel.”

### (Measurement of Weight of Sample of Dried Hydrogel)

Weights of the prepared four samples were measured. The weight measurement was performed after 0 hour, 1 hour, 2 hours, 4 hours, 8 hours, 24 hours, 48 hours, 72 hours, 120 hours, and 168 hours from the disposition of the sample of the hydrogel compositions into the incubator.

FIG. 4 is a graph showing a change over time in weight in a sample of a dried hydrogel according to Example 3 of the present invention. The horizontal axis of the graph in FIG. 4 shows elapsed time from the disposition of the sample into the incubator and the unit is hour (h). The longitudinal axis of the graph in FIG. 4 shows the weight of the sample and the unit is gram (g).

As shown in FIG. 4, after 72 hours from the disposition of the sample of the hydrogel composition into the incubator, the weight of the sample became almost constant. Specifically, in the case of representing the weight of the sample as average value±standard deviation, the weight of the sample after 72 hours was 0.108±0.006 (g). The weight of the sample after 120 hours was 0.104±0.005 (g). Further, the weight of the sample after 168 hours was 0.099±0.005 (g)

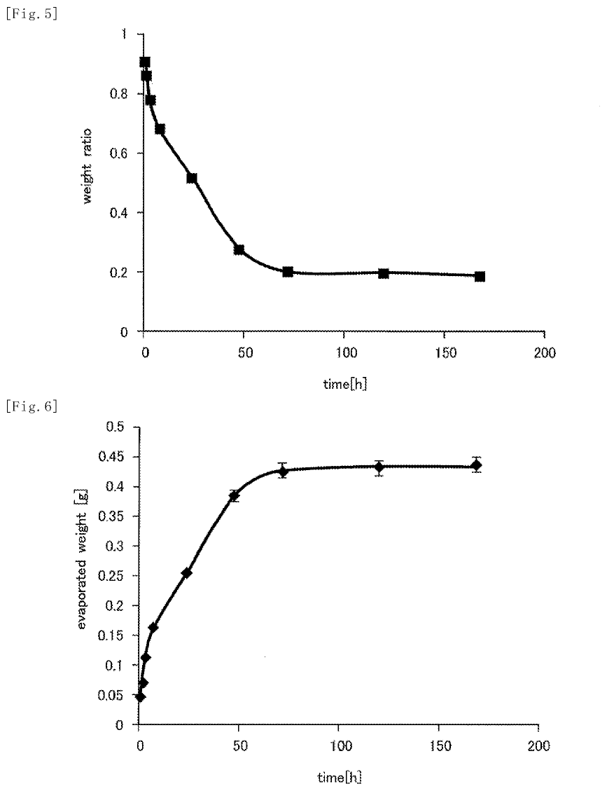

FIG. 5 is a graph showing a change over time in weight ratio in the sample of the dried hydrogel according to Example 3 of the present invention with respect to a sample of a hydrogel composition. The horizontal axis of the graph in FIG. 5 shows elapsed time from the disposition of the sample into the incubator and the unit is hour (h). The longitudinal axis of the graph in FIG. 5 shows the weight ratio of the sample in a case where the weight of the sample of the hydrogel composition is regarded as 1. That is, the weight ratio of the sample of the dried hydrogel was calculated by dividing the weight of the sample of the dried hydrogel by the weight of the sample of the hydrogel composition.

As shown in FIG. 5, after 72 hours from the disposition of the sample of the hydrogel composition into the incubator, the weight ratio of the sample became almost constant. Specifically, in the case of representing the weight ratio of the sample as average value±standard deviation, the weight ratio of the sample after 72 hours was 0.201±0.004. The weight ratio of the sample after 120 hours became 0.192±0.002. Further, the weight ratio of the sample after 168 hours became 0.184±0.003.

Taking the ratio of the weight of the mixed solvent of dimethyl sulfoxide and water in the sample of the hydrogel composition into consideration, the lower limit of the weight ratio described above is considered to be 0.15. Therefore, from the results of FIG. 5, it was shown that the weight ratio of the dried hydrogel used in the dried tissue model to the hydrogel composition can be set to preferably 0.15 to 0.21.

FIG. 6 is a graph showing a change over time in solvent evaporated weight in the sample of the dried hydrogel according to Example 3 of the present invention. The horizontal axis of the graph in FIG. 6 shows elapsed time from the disposition of the sample into the incubator and the unit is hour (h). The longitudinal axis of the graph in FIG. 6 shows a solvent evaporated weight from the sample of the hydrogel composition and the unit is gram (g). Incidentally, the solvent evaporated weight was calculated by subtracting the weight of the dried hydrogel from the weight of the sample of the hydrogel composition.

As shown in FIG. 6, after 72 hours from the disposition of the sample of the hydrogel composition into the incubator, the solvent evaporated weight became almost constant. Specifically, in the case of representing the solvent evaporated weight of the sample as average value±standard deviation, the solvent evaporated weight after 72 hours became 0.427±0.013 (g). The solvent evaporated weight after 120 hours became 0.431±0.014 (g). Further, the solvent evaporated weight after 168 hours became 0.436±0.014 (g).

FIG. 7 is a graph showing a change over time in solvent evaporated weight per hour in the sample of the dried hydrogel according to Example 3 of the present invention. The horizontal axis of the graph in FIG. 7 shows elapsed time from the disposition of the sample into the incubator and the unit is hour (h). The longitudinal axis of the graph in FIG. 7 shows a solvent evaporated weight per hour and the unit is gram/hour (g/h). Incidentally, the solvent evaporated weight per hour was calculated by dividing a difference value of the solvent evaporated weight by a difference value of hour.

As shown in FIG. 7, after 72 hours from the disposition of the sample of the hydrogel composition into the incubator, the solvent evaporated weight per hour became almost 0. Specifically, in the case of representing the solvent evaporated weight per hour as average value±standard deviation, the solvent evaporated weight per hour after 72 hours became 0.173×10⁻²±0.430×10⁻³ (g/h). The solvent evaporated weight per hour after 120 hours became 0.938×10⁻⁶±0.333×10⁻⁶ (g/h). Further, the solvent evaporated weight per hour after 168 hours became 0.990×10⁻⁶±0.598×10⁻⁷ (g/h).

As shown in graphs of FIGS. 4 to 7, in a case where the dried hydrogel is dried at a temperature of 40° C., the solvent evaporated weight is saturated after 72 hours. Therefore, it was shown that a dried tissue model can be prepared by being dried at a temperature of 40° C. for 72 hours.

### Example 4

### (Preparation of Sample of Impregnated Hydrogel)

The four samples of the dried hydrogel prepared in Example 3 were immersed in water set to 25° C., 30° C., 35° C., and 40° C. for 3 hours. The temperatures of the water were maintained to be constant at 25° C., 30° C., 35° C., and 40° C.

A solvent with which the dried hydrogel can be impregnated corresponds to a “second solvent,” and water in Example 4 is an example of the second solvent. In addition, the temperature of the second solvent with which the dried hydrogel is impregnated is referred to as a “third temperature” in some cases. Further, a product produced by impregnated the dried hydrogel with the second solvent is referred to as an “impregnated hydrogel.”

### (Measurement of Weight of Sample of Impregnated Hydrogel)

Weights of the four samples were measured. The weight measurement was performed after 0 hour, 1 hour, 2 hours, and 3 hours from the start of immersion of the samples.

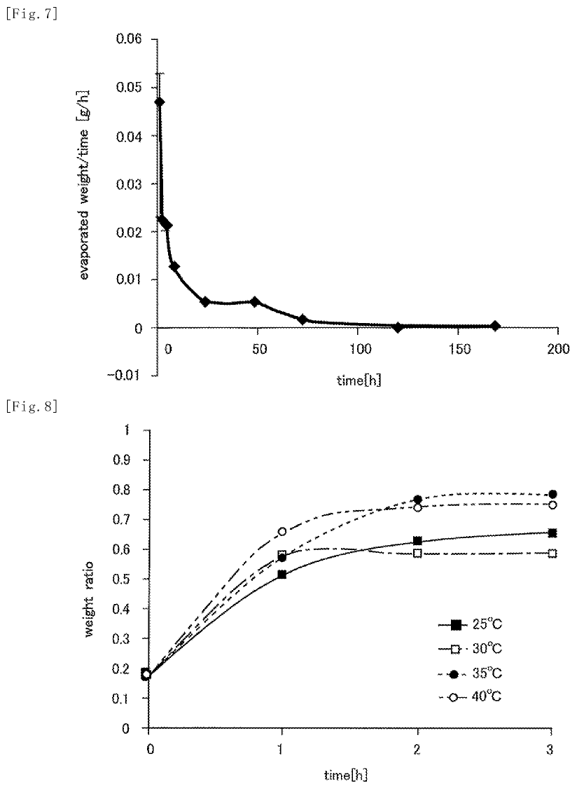

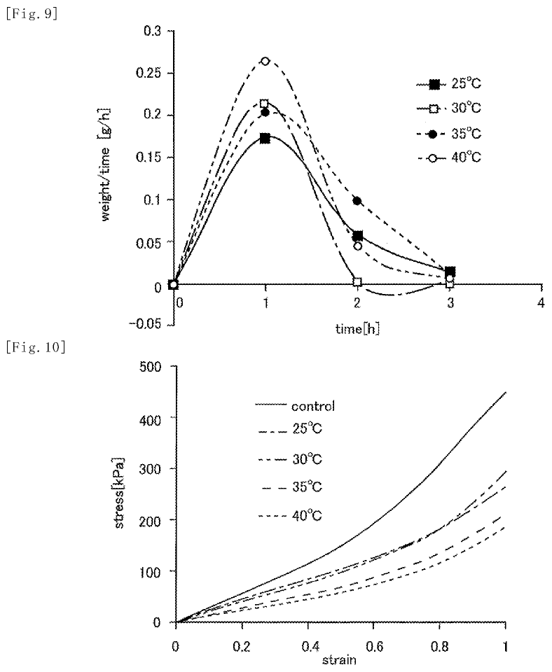

FIG. 8 is a graph showing a change over time in weight ratio in a sample of an impregnated hydrogel according to Example 4 of the present invention with respect to a sample of a hydrogel composition. The horizontal axis of the graph in FIG. 8 shows elapsed time from the start of immersion of the sample and the unit is hour (h). The longitudinal axis of the graph in FIG. 8 shows a weight ratio of the sample in a case where the weight of the sample of the hydrogel composition is regarded as 1. That is, the weight ratio of the sample of the impregnated hydrogel was calculated by dividing the weight of the sample of the impregnated hydrogel by the weight of the sample of the hydrogel composition. In FIG. 8, a change in weight ratio of the sample in the case of the immersion in water set at 25° C. is shown by black square blotting and the solid curve. In addition, in FIG. 8, a change in weight ratio of the sample in the case of the immersion in water set at 30° C. is shown by white square blotting and the dashed-dotted line curve. Further, in FIG. 8, a change in weight ratio of the sample in the case of the immersion in water set at 35° C. is shown by black circle blotting and the dotted line curve. Furthermore, in FIG. 8, a change in weight ratio of the sample in the case of the immersion in water set at 40° C. is shown by white circle blotting and the dashed-two dotted line curve.

As shown in FIG. 8, the weight ratio of the sample after 3 hours in the case of the temperature of water being 25° C. became 0.657. Further, the weight ratio of the sample after 3 hours in the case of the temperature of water being 30° C. became 0.587. Furthermore, the weight ratio of the sample after 3 hours in the case of the temperature of water being 35° C. became 0.785. Furthermore, the weight ratio of the sample after 3 hours in the case of the temperature of water being 40° C. became 0.752. From the above-described results, it can be said that the weight ratio of the impregnated hydrogel to the hydrogel composition can be adjusted to preferably 0.55 to 0.80 by the immersion in the second solvent.

FIG. 9 is a graph showing a change over time in weight per hour in the sample of the impregnated hydrogel according to Example 4 of the present invention. The horizontal axis of the graph in FIG. 9 shows elapsed time from the start of immersion of the sample and the unit is hour (h). The longitudinal axis of the graph in FIG. 9 shows a solvent impregnation weight per hour and the unit is gram/hour (g/h). Incidentally, the solvent impregnation weight per hour was calculated by dividing a difference value of the solvent impregnation weight by a difference value of hour. In FIG. 9, a change in solvent impregnation weight per hour in the case of the immersion in water set at 25° C. is shown by black square blotting and the solid curve. In addition, in FIG. 9, a change in solvent impregnation weight per hour in the case of the immersion in water set at 30° C. is shown by white square blotting and the dashed-dotted line curve. Further, in FIG. 9, a change in solvent impregnation weight per hour in the case of the immersion in water set at 35° C. is shown by black circle blotting and the dotted line curve. Furthermore, in FIG. 9, a change in solvent impregnation weight per hour in the case of the immersion in water set at 40° C. is shown by white circle blotting and the dashed-two dotted line curve.

As shown in FIG. 9, after 3 hours from the start of immersion of the sample, the solvent impregnation weight per hour became almost 0 at all temperatures. Specifically, the solvent impregnation weight per hour in the case of immersing the sample in water set at 25° C. became 0.015 (g/h) after 3 hours. In addition, the solvent impregnation weight per hour in the case of immersing the sample in water set at 30° C. became 0.001 (g/h) after 3 hours. Further, the solvent impregnation weight per hour in the case of immersing the sample in water set at 35° C. became 0.010 (g/h) after 3 hours. Furthermore, the solvent impregnation weight per hour in the case of immersing the sample in water set at 40° C. became 0.008 (g/h) after 3 hours.

As shown in FIG. 8, it was shown that by the immersion in the second solvent, the impregnated hydrogel can be prepared such that the weight ratio of the impregnated hydrogel to the hydrogel composition becomes 0.55 to 0.80 and it was shown that the impregnated hydrogel can be configured to have a desired weight by the immersion in the second solvent. That is, in Example 4, it was shown that, by immersing the dried tissue model in the solvent, a tissue model including an impregnated hydrogel having a desired weight can be remanufactured.

Further, in the graph of FIG. 9, it was shown that in the case of immersing the dried hydrogel in the second solvent set at a temperature of 25° C. to 40° C., the solvent impregnation weight is saturated after 3 hours. Therefore, in Example 4, it was shown that, by a simple method of immersion in the second solvent set at a temperature of 25° C. to 40° C. for 3 hours or longer, a tissue model having a desired weight can be manufactured from the dried tissue model.

### Example 5

### (Measurement of Tensile Stress of Impregnated Hydrogel)

The tensile stress of the sample of the impregnated hydrogel obtained in Example 4 was measured. In measurement of the tensile stress of the sample of the hydrogel composition, similarly to Example 2 described above, a small table-top testing machine, Model No. EZ-S, manufactured by SHIMADZU CORPORATION was used. Both ends at the long side of the sample of the impregnated hydrogel were held with grips of the uniaxial tensile testing machine to fix the sample of the impregnated hydrogel.

Next, the fixed sample of the impregnated hydrogel was pulled in both directions at a speed of 20 mm/min until the strain of the sample of the impregnated hydrogel would be 100%, and then the strain of the sample of the impregnated hydrogel was restored to 0%. That is, the fixed sample of the impregnated hydrogel was pulled in both directions until the distance between the grips would be two times the initial distance, and then the distance between the grips was restored to the initial distance.

In Example 5, similarly to Example 2 described above, taking hysteresis of the sample of the impregnated hydrogel into consideration, the above-described operation cycle was repeated three times, and the strain of the sample of the impregnated hydrogel and the tensile stress of the sample of the impregnated hydrogel in the third cycle were measured for every 0.05 seconds.

FIG. 10 is a graph showing a stress-strain curve of the hydrogel composition and the impregnated hydrogel of the present invention. The horizontal axis of the graph in FIG. 10 shows strain, the lower limit is 0, and the upper limit is 1. The longitudinal axis of the graph in FIG. 10 shows tensile stress and the unit is kilopascal (kPa). In FIG. 10, as the curve of a control value, the stress-strain curve of the sample of the hydrogel composition is represented by the solid line. Further, in FIG. 10, the stress-strain curve of the sample of the impregnated hydrogel impregnated with water set at 25° C. is represented by the dashed-dotted line. Furthermore, in FIG. 10, the stress-strain curve of the sample of the impregnated hydrogel impregnated with water set at 30° C. is represented by the dashed-two dotted line. Furthermore, in FIG. 10, the stress-strain curve of the sample of the impregnated hydrogel impregnated with water set at 35° C. is represented by the dashed line. Furthermore, in FIG. 10, the stress-strain curve of the sample of the impregnated hydrogel impregnated with water set at 40° C. is represented by the dotted line. As shown in FIG. 10, in the samples of the hydrogel composition and the impregnated hydrogel, when the strain value is larger than 0.6, non-linearity between the strain and the tensile stress becomes significant.

Values of tensile stress of the sample of the impregnated hydrogel impregnated with water set at 25° C. with respect to strains of 25%, 50%, 75%, and 100% are shown in the following Table 2.

TABLE 2

| Strain (ΔL/L) | 0.25 | 0.50 | 0.75 | 1.00 |
|---|---|---|---|---|
| Stress (kPa) | 64.0 | 146.0 | 274.1 | 447.5 |

Values of tensile stress of the sample of the impregnated hydrogel impregnated with water set at 30° C. with respect to strains of 25%, 50%, 75%, and 100% are shown in the following Table 3.

TABLE 3

| Strain (ΔL/L) | 0.25 | 0.50 | 0.75 | 1.00 |
|---|---|---|---|---|
| Stress (kPa) | 46.7 | 95.8 | 163.6 | 291.6 |

Values of tensile stress of the sample of the impregnated hydrogel impregnated with water set at 35° C. with respect to strains of 25%, 50%, 75%, and 100% are shown in the following Table 4.

TABLE 4

| Strain (ΔL/L) | 0.25 | 0.50 | 0.75 | 1.00 |
|---|---|---|---|---|
| Stress (kPa) | 32.4 | 67.6 | 118.8 | 213.2 |

Values of tensile stress of the sample of the impregnated hydrogel impregnated with water set at 40° C. with respect to strains of 25%, 50%, 75%, and 100% are shown in the following Table 5.

TABLE 5

| Strain (ΔL/L) | 0.25 | 0.50 | 0.75 | 1.00 |
|---|---|---|---|---|
| Stress (kPa) | 27.1 | 57.2 | 101.7 | 188.5 |

In Tables 2 to 5, a strain ΔL/L is represented by the ratio of elongation ΔL of the sample of the hydrogel composition to an initial distance L between the grips. That is, values of strains of “0.25,” “0.5,” “0.75,” and “1” in Tables 2 to 5 respectively correspond to strains of 25%, 50%, 75%, and 100%. In addition, in Tables 2 to 5, the tensile stress was represented by kilopascal (kPa) unit, and was calculated from measurement values of tensile stress corresponding to measurement values of strains best approximation to strains of 25%, 50%, 75%, and 100%.

Further, the Young's modulus that is an index for an elasticity in tension of the sample of the impregnated hydrogel was calculated from the results of the tensile test. As described above, in the samples of the hydrogel composition and the impregnated hydrogel, when the strain value is larger than 0.6, non-linearity between the strain and the tensile stress becomes significant. Therefore, regarding the sample of the impregnated hydrogel, the Young's modulus was calculated as a Young's modulus at a strain of 50% in which linearity of the Young's modulus is maintained.

The Young's modulus of the sample of the impregnated hydrogel impregnated with water set at 25° C. at a strain of 50% was 200 (kPa). The Young's modulus of the sample of the impregnated hydrogel impregnated with water set at 30° C. at a strain of 50% was 192 (kPa). The Young's modulus of the sample of the impregnated hydrogel impregnated with water set at 35° C. at a strain of 50% was 136 (kPa). The Young's modulus of the sample of the impregnated hydrogel impregnated with water set at 40° C. at a strain of 50% was 115 (kPa).

Incidentally, the Young's modulus of the impregnated hydrogel at a strain of 50% corresponds to a “second Young's modulus.”

The ratio of the second Young's modulus to the first Young's modulus of the hydrogel composition described in Example 2 was calculated by using the second Young's modulus of the impregnated hydrogel described above, and then an index for properties of the dried hydrogel included in the dried tissue model was determined. The ratio was calculated by dividing the second Young's modulus by the first Young's modulus.

The ratio of the second Young's modulus to the first Young's modulus of the impregnated hydrogel impregnated with water set at 25° C. was 0.69. The ratio of the second Young's modulus to the first Young's modulus of the impregnated hydrogel impregnated with water set at 30° C. was 0.66. The ratio of the second Young's modulus to the first Young's modulus of the impregnated hydrogel impregnated with water set at 35° C. was 0.47. The ratio of the second Young's modulus to the first Young's modulus of the impregnated hydrogel impregnated with water set at 40° C. was 0.39.

Therefore, the dried tissue model can be configured by using a dried hydrogel in which the ratio of the second Young's modulus at a strain of 0.5 of the impregnated hydrogel to the first Young's modulus at a strain of 0.5 of the hydrogel composition is 0.39 to 0.69. That is, in Example 5, it was shown that a dried tissue model can be configured such that a tissue model having a desired stress is obtained and it is possible to remanufacture a tissue model including an impregnated hydrogel having a desired stress by the immersion in the solvent.

### Example 6

### (Measurement of Elasticity in Shear and Viscosity of Impregnated Hydrogel)

The dynamic viscoelasticity, that is, the modulus of elasticity in shear and the viscosity of the sample of the impregnated hydrogel were measured. Four test pieces of the hydrogel composition were prepared by the method described in Example 1 using a square mold having a size of about 8 mm×8 mm and a thickness of about 1 mm. The prepared four test pieces of the hydrogel composition were dried by the method described in Example 3 to prepare four test pieces of the dried hydrogel. The prepared four test pieces of the dried hydrogel were respectively immersed in water set at 25° C., 30° C., 35° C., and 40° C. by the method described in Example 4 to prepare four test pieces of the impregnated hydrogel. The test piece of the impregnated hydrogel impregnated with water set at 25° C. had a size of 7.4 mm×7.4 mm and a thickness of 0.87 mm. The test piece of the impregnated hydrogel impregnated with water set at 30° C. had a size of 7.5 mm×7.5 mm and a thickness of 0.87 mm. The test piece of the impregnated hydrogel impregnated with water set at 35° C. had a size of 7.8 mm×7.8 mm and a thickness of 0.92 mm. The test piece of the impregnated hydrogel impregnated with water set at 40° C. had a size of 8.0 mm×8.0 mm and a thickness of 0.94 mm.

In measurement of the modulus of elasticity in shear and the viscosity of the prepared test pieces, a dynamic viscoelastic device, Model No. DMS6100, manufactured by SII NanoTechnology Inc. was used. The prepared test piece was fixed to the dynamic viscoelastic device, the temperature of the test piece was increased from room temperature at a temperature increase rate of 2° C./min, and then the test piece was caused to vibrate by applying minute sinusoidal vibration having a frequency of 1 Hz and an amplitude of 10±0.5 μm. Under the vibration of the test piece, a storage elastic modulus that is an index for the modulus of elasticity in shear and a loss elastic modulus that is an index for the viscosity were measured by automated sampling along with a temperature of the test piece. The storage elastic modulus and the loss elastic modulus were measured. In Example 6, a storage elastic modulus when the temperature of the test piece is best approximation to 24° C. was regarded as an index value of the modulus of elasticity in shear. Similarly, a loss elastic modulus when the temperature of the test piece is best approximation to 24° C. was regarded as an index value of the viscosity. That is, index values of the modulus of elasticity in shear and the viscosity were calculated from measurement values of the storage elastic modulus and the loss elastic modulus, respectively.

The measurement of the modulus of elasticity in shear and the viscosity by the dynamic viscoelastic device is usually performed, as described above, by applying sinusoidal vibration to a certain direction. Meanwhile, in the dynamic viscoelastic device, in order to avoid that the strain of the test piece caused by vibration becomes too large, a direction of the vibration applied to the test piece is changed to a direction perpendicular to a usual direction in some cases. Therefore, since there is a case where the storage elastic modulus and the loss elastic modulus when the test piece vibrates in a different direction are measured in the dynamic viscoelastic device, it is necessary to correct index values of the modulus of elasticity in shear and the viscosity and calculate values as actual values of a modulus of elasticity in shear G′ and a viscosity G″.

The modulus of elasticity in shear G′ and the viscosity G″ are calculated by dividing index values of the modulus of elasticity in shear and the viscosity by the form factor coefficient α. In the case of measuring the modulus of elasticity in shear and the viscosity, the form factor coefficient α depends on a cross-sectional area S (mm²) of the sample in a direction perpendicular to a direction to which vibration is applied and a length L (mm) of the sample in the direction to which vibration is applied, and is defined by the following formula.

a=(2×S)/(1000×L) (5)

In the case of the test piece prepared in Example 6, the cross-sectional area S corresponds to an area of the test piece and the length L corresponds to a thickness of the test piece. That is, in the test piece of the impregnated hydrogel impregnated with water set at 25° C., the cross-sectional area S becomes 7.4×7.4 mm², and the length L becomes 0.87 mm. In the test piece of the impregnated hydrogel impregnated with water set at 30° C., the cross-sectional area S becomes 7.5×7.5 mm², and the length L becomes 0.87 mm. In the test piece of the impregnated hydrogel impregnated with water set at 35° C., the cross-sectional area S becomes 7.8×7.8 mm², and the length L becomes 0.92 mm. In the test piece of the impregnated hydrogel impregnated with water set at 40° C., the cross-sectional area S becomes 8.0×8.0 mm², and the length L becomes 0.94 mm.

Therefore, the form factor coefficient α of the test piece of the impregnated hydrogel impregnated with water set at 25° C. was calculated to be 0.125. Further, the form factor coefficient α of the test piece of the impregnated hydrogel impregnated with water set at 30° C. was calculated to be 0.129. Furthermore, the form factor coefficient α of the test piece of the impregnated hydrogel impregnated with water set at 35° C. was calculated to be 0.132. Furthermore, the form factor coefficient α of the test piece of the impregnated hydrogel impregnated with water set at 40° C. was calculated to be 0.137.

FIG. 11 is a graph showing a relation between a temperature and a dynamic viscoelasticity in an impregnated hydrogel according to Example 6 of the present invention. The horizontal axis of the graph in FIG. 11 shows temperature and the unit is degree Celsius (° C.). The left longitudinal axis of the graph in FIG. 11 shows the modulus of elasticity in shear G′ and the unit is kilopascal (kPa). The right longitudinal axis of the graph in FIG. 11 shows a viscosity G″ and the unit is kilopascal (kPa). In FIG. 11, the modulus of elasticity in shear G′ of each of the samples of the impregnated hydrogel respectively impregnated with water set at 25° C., 30° C., 35° C., and 40° C. was shown by black rhombic blotting.

The index value of the modulus of elasticity in shear of the impregnated hydrogel impregnated with water set at 25° C. as measured by a dynamic viscoelastic device was 2.5×10⁴ (Pa). Further, the index value of the modulus of elasticity in shear of the impregnated hydrogel impregnated with water set at 30° C. was 2.7×10⁴ (Pa). Furthermore, the index value of the modulus of elasticity in shear of the impregnated hydrogel impregnated with water set at 35° C. was 2.0×10⁴ (Pa). Furthermore, the index value of the modulus of elasticity in shear of the impregnated hydrogel impregnated with water set at 40° C. was 1.2×10⁴ (Pa).

When the value of the modulus of elasticity in shear G′ is calculated by dividing the index value of the modulus of elasticity in shear described above by the form factor coefficient, the modulus of elasticity in shear G′ of the impregnated hydrogel impregnated with water set at 25° C. becomes 2.0×10² (kPa). Further, the modulus of elasticity in shear G′ of the impregnated hydrogel impregnated with water set at 30° C. becomes 2.1×10² (kPa). Furthermore, the modulus of elasticity in shear G′ of the impregnated hydrogel impregnated with water set at 35° C. becomes 1.5×10² (kPa). Furthermore, the modulus of elasticity in shear G′ of the impregnated hydrogel impregnated with water set at 40° C. becomes 0.9×10² (kPa).

Therefore, the dried tissue model can be configured by using a dried hydrogel in which the modulus of elasticity in shear G′ of the impregnated hydrogel becomes 0.9×10² to 2.1×10² (kPa). That is, in Example 6, it was shown that a dried tissue model can be configured such that a tissue model having a desired elasticity in shear G′ is obtained, and by the immersion in the solvent, it is possible to remanufacture a tissue model including an impregnated hydrogel having a desired elasticity in shear G′.

Further, as shown in FIG. 11, regarding the modulus of elasticity in shear G′ of the impregnated hydrogel, in a temperature range of 30 to 40° C., the decrease rate of the modulus of elasticity in shear G′ with respect to an increase in temperature of water used for immersion became constant. Therefore, it was shown that, by performing immersion while a temperature of the second solvent is adjusted between 30 to 40° C., it is possible to easily remanufacture a tissue model including an impregnated hydrogel having a different elasticity in shear G′. Further, it was shown that, even in the case of the tissue model being dried, by performing immersion while a temperature of the second solvent is adjusted between 30 to 40° C., it is possible to remanufacture a tissue model having a desired elasticity in shear G′ in a reusable manner.

In FIG. 11, the viscosities G″ of each of the samples of the impregnated hydrogel respectively impregnated with water set at 25° C., 30° C., 35° C., and 40° C. were shown by black square blotting.

The index value of the viscosity of the impregnated hydrogel impregnated with water set at 25° C. as measured by a dynamic viscoelastic device was 2.2×10³ (Pa). Further, the index value of the viscosity of the impregnated hydrogel impregnated with water set at 30° C. was 2.1×10³ (Pa). Further, the index value of the viscosity of the impregnated hydrogel impregnated with water set at 35° C. was 1.4×10³ (Pa). Further, the index value of the viscosity of the impregnated hydrogel impregnated with water set at 40° C. was 0.66×10³ (Pa).

When a value of the viscosity G″ is calculated by dividing the above-described index value of the viscosity by the form factor coefficient, the viscosity G″ of the impregnated hydrogel impregnated with water set at 25° C. becomes 17.6 (kPa). Further, the viscosity G″ of the impregnated hydrogel impregnated with water set at 30° C. becomes 16.3 (kPa). Further, the viscosity G″ of the impregnated hydrogel impregnated with water set at 35° C. becomes 10.6 (kPa). Further, the viscosity G″ of the impregnated hydrogel impregnated with water set at 40° C. becomes 4.8 (kPa).

Therefore, the dried tissue model can be configured by using a dried hydrogel in which the viscosity G″ of the impregnated hydrogel becomes 4.8 to 17.6 (kPa). That is, in Example 6, it was shown that a dried tissue model can be configured such that a tissue model having a desired viscosity G″ is obtained, and by the immersion in the solvent, it is possible to remanufacture a tissue model including an impregnated hydrogel having a desired viscosity G″.

Further, as shown in FIG. 11, regarding the viscosity G″ of the impregnated hydrogel, in a temperature range of to 40° C., the decrease rate of the viscosity G″ with respect to an increase in temperature of water used for immersion became constant. Therefore, it was shown that, by performing immersion while a temperature of the second solvent is adjusted between 30 to 40° C., it is possible to easily remanufacture a tissue model including an impregnated hydrogel having a different viscosity G″. Further, it was shown that, even in the case of the tissue model being dried, by performing immersion while a temperature of the second solvent is adjusted between 30 to 40° C., it is possible to remanufacture a tissue model having a desired viscosity G″ in a reusable manner.

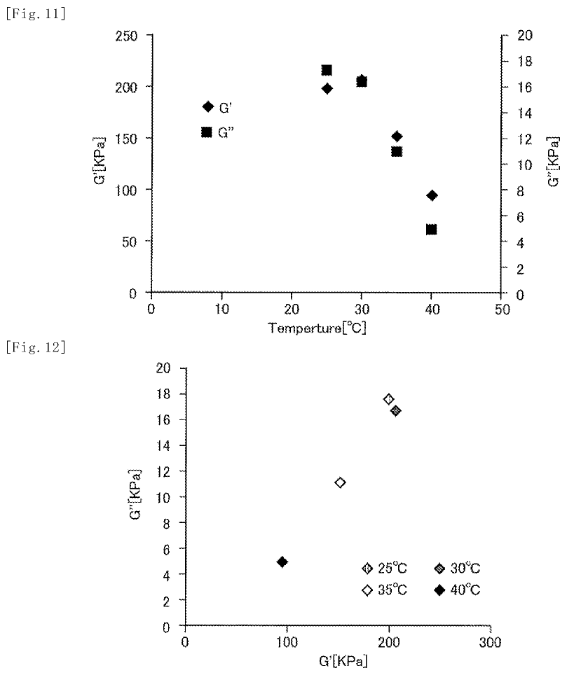

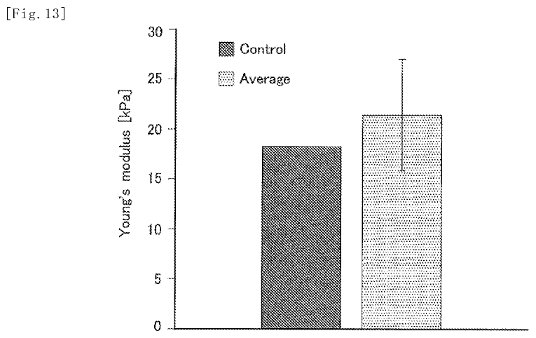

FIG. 12 is a graph showing a correlation of a dynamic viscoelasticity in the impregnated hydrogel according to Example 6 of the present invention. The horizontal axis of the graph in FIG. 12 shows the modulus of elasticity in shear G′ and the unit is pascal (Pa). The longitudinal axis of the graph in FIG. 12 shows a viscosity G″ and the unit is pascal (Pa). In FIG. 12, the modulus of elasticity in shear G′ and the viscosity G″ of the sample of the impregnated hydrogel impregnated with water set at 25° C. were shown by vertically striped rhombic blotting. Further, in FIG. 12, the modulus of elasticity in shear G′ and the viscosity G″ of the sample of the impregnated hydrogel impregnated with water set at 30° C. were shown by checked rhombic blotting. Further, in FIG. 12, the modulus of elasticity in shear G′ and the viscosity G″ of the sample of the impregnated hydrogel impregnated with water set at 35° C. were shown by white rhombic blotting. Further, in FIG. 12, the modulus of elasticity in shear G′ and the viscosity G″ of the sample of the impregnated hydrogel impregnated with water set at 40° C. were shown by black rhombic blotting.

As shown in FIG. 12, in a range in which the temperature of water with which the hydrogel is impregnated is 30 to 40° C., the modulus of elasticity in shear G′ and the viscosity G″ had a linear relation. Therefore, in Example 6, it was shown that, by appropriately setting the temperature of the second solvent in a temperature range of 30 to 40° C., a dried tissue model can be configured such that a tissue model having a desired elasticity in shear G′ and a desired viscosity G″ is easily obtained. Further, it was shown that, even in the case of the tissue model being dried, by performing immersion while a temperature of the second solvent is adjusted between 30 to 40° C., it is possible to remanufacture a tissue model having a desired elasticity in shear G′ and a desired viscosity G″ in a reusable manner.

### Example 7

(Measurement of Young's Modulus of Impregnated Hydrogel after Repetition of Drying and Immersing of Hydrogel Composition)

Two samples of a hydrogel composition were prepared by the method described in Example 1. One of the samples was disposed in an incubator, and the mixed solvent of dimethyl sulfoxide and water contained in the sample of the hydrogel composition was vaporized inside the incubator for 5 hours, thereby preparing a sample of a dried hydrogel. The internal temperature of the incubator was maintained to 40° C. The prepared sample of the dried hydrogel was immersed in water set at 35° C. for 3 hours to prepare a sample of an impregnated hydrogel. The temperature of water was maintained to be constant at 35° C. using a heater. In Example 7, the above-described cycle was repeated three times to prepare the sample of the impregnated hydrogel three times as a remanufactured product of the hydrogel composition.

The other of the samples was immersed in pure water for 24 hours to prepare a sample of a hydrogel composition in which dimethyl sulfoxide (DMSO) contained in the hydrogel composition is replaced by water.

The tensile stress of the prepared sample described above was measured using a small table-top testing machine, Model No. EZ-S, manufactured by SHIMADZU CORPORATION, similarly to Example 2 and Example 5 described above. Further, similarly to Example 2 and Example 5 described above, the Young's modulus that is an index for an elasticity in tension of the sample was calculated from the measurement results of the tensile stress at a strain of 50%. Incidentally, the tensile stress at a stain of 50% was calculated from a measurement value of tensile stress corresponding to a measurement value of a strain best approximation to a strain of 50%.

The Young's modulus of the sample of the hydrogel composition was 18.3 (kPa). Meanwhile, the Young's modulus of the sample of the impregnated hydrogel remanufactured at the first cycle was 15.3 (kPa). Further, the Young's modulus of the sample of the impregnated hydrogel remanufactured at the second cycle was 26.8 (kPa). Further, the Young's modulus of the sample of the impregnated hydrogel remanufactured at the third cycle was 25.3 (kPa). Further, an average value of Young's moduli of the samples of the impregnated hydrogel was 22.4±6.3 (kPa) in the case of being represented as average value±standard deviation.

FIG. 13 is a graph showing an average value of Young's moduli in repeated remanufacturing of an impregnated hydrogel according to Example 7 of the present invention. The Young's modulus of the sample of the hydrogel composition is shown in a bar graph at the left side of the graph of FIG. 13. The average value and the standard deviation of Young's moduli of the sample of the impregnated hydrogel are shown in a bar graph with an error bar at the right side of the graph of FIG. 13. The longitudinal axis of the graph in FIG. 13 shows a Young's modulus and the unit is kilopascal (kPa).

As shown in FIG. 13, when compared to the sample of the hydrogel composition, the sample of the impregnated hydrogel tends to harden, but a difference in Young's modulus therebetween is about 4.1 (kPa), and thus this is considered to be an allowable range for withstanding use as a tissue model. Therefore, according to the dried tissue model of the present invention, even in a case where the model is immersed in water and then dried again, the model can be restored to have a Young's modulus in an allowable range by being immersed again in water, and thus the dried tissue model can be repeatedly reused. From the above description, in the present invention, it is possible to provide a dried tissue model by which convenience of use of a tissue model can be improved and time and cost for manufacturing a new tissue model can be reduced.

Incidentally, in the above-described Examples, the second solvent in which the dried hydrogel is immersed is water, but is not limited thereto. For example, as the second solvent, a mixed solvent of water and an organic solvent which is miscible in water, water, or saline can also be used.

### REFERENCE SIGNS LIST

1 Polyvinyl alcohol
2 Basic skeleton
2a First hydrocarbon skeleton part
2b Second hydrocarbon skeleton part
3 Functional group
4 Acetate group
5 Hydroxyl group
10 Hydrogel composition
12 Polymer chain
12a First polymer chain
12b Second polymer chain
14 Crosslink region
16 Network portion
18 Solvent

1. A dried tissue model, comprising:
a dried hydrogel which is a dried product of a hydrogel composition, the hydrogel composition comprising
a polyvinyl alcohol resin having a three-dimensional network structure, and
a first solvent which is confined in a network portion of the three-dimensional network structure to lose fluidity,
wherein when a solvent with which the dried hydrogen can be impregnated is used as a second solvent and the dried hydrogel is impregnated with the second solvent to form an impregnated hydrogel, the impregnated hydrogel has a modulus of elasticity in shear of 0.9×10² to 2.1×10² kPa.

2. The dried tissue model according to claim 1, wherein in a range in which a temperature of the second solvent is 30 to 40° C.,
a decrease rate of the modulus of elasticity in shear with respect to an increase in temperature of the second solvent is constant.

3. The dried tissue model according to claim 1 or 2, wherein a viscosity of the impregnated hydrogel is 4.8 to 17.6 kPa.

4. The dried tissue model according to claim 3, wherein in a range in which a temperature of the second solvent is 30 to 40° C.,
a decrease rate of the viscosity with respect to an increase in temperature of the second solvent is constant.

5. The dried tissue model according to claim 3, wherein in a range in which a temperature of the second solvent is 30 to 40° C.,
the modulus of elasticity in shear and the viscosity have a linear relation.

6. A dried tissue model, comprising:
a dried hydrogel which is a dried product of a hydrogel composition, the hydrogel composition comprising
a polyvinyl alcohol resin having a three-dimensional network structure, and
a first solvent which is confined in a network portion of the three-dimensional network structure to lose fluidity,
wherein when a solvent with which the dried hydrogel can be impregnated is used as a second solvent and the dried hydrogel is impregnated with the second solvent to form an impregnated hydrogel, the impregnated hydrogel has a viscosity of 4.8 to 17.6 kPa.

7. The dried tissue model according to claim 6, wherein in a range in which a temperature of the second solvent is 30 to 40° C.,
a decrease rate of the viscosity with respect to an increase in temperature of the second solvent is constant.

8. The dried tissue model according to claim 1, wherein the ratio of a second Young's modulus at a strain of 0.5 of the impregnated hydrogel to a first Young's modulus at a strain of 0.5 of the hydrogel composition is 0.39 to 0.69.

9. A dried tissue model, comprising:
a dried hydrogel which is a dried product of a hydrogel composition, the hydrogel composition comprising
a polyvinyl alcohol resin having a three-dimensional network structure, and
a first solvent which is confined in a network portion of the three-dimensional network structure to lose fluidity,
wherein when a solvent with which the dried hydrogel can be impregnated is used as a second solvent and the dried hydrogel is impregnated with the second solvent to form an impregnated hydrogel, the ratio of a second Young's modulus at a strain of 0.5 of the impregnated hydrogel to a first Young's modulus at a strain of 0.5 of the hydrogel composition is 0.39 to 0.69.

10. The dried tissue model according to claim 8, wherein a stress at a strain of 0.25 to 0.5 of the hydrogel composition is 64.0 to 146.0 kPa.

11-24. (canceled)

25. The dried tissue model according to claim 1, wherein the tissue is a body soft tissue.

26. The dried tissue model according to claim 25, wherein the body soft tissue is a vascularized tissue.

27-30. (canceled)
